(19) United States
(12) Patent Application Publication
(10) Pub. No.: US 2025/0299821 A1
(43) Pub. Date: Sep. 25, 2025

(54) PERCENTAGE STENOSIS OF THE ARTERIES OF THE HEART: CAUSES, OPTIMIZATION, METHOD, AND PROBABILISTIC STRATEGIES

(71) Applicant: UNIVERSITY OF SOUTH FLORIDA, Tampa, FL (US)

(72) Inventors: Chris TSOKOS, Tampa, FL (US); Erasmus TETTEH-BATOR, Tampa, FL (US)

(21) Appl. No.: 19/087,380

(22) Filed: Mar. 21, 2025

Related U.S. Application Data
(60) Provisional application No. 63/567,973, filed on Mar. 21, 2024.

(51) Int. Cl. G16H 50/20; G16H 10/60; G16H 20/00

(52) U.S. Cl. CPC G16H 50/20; G16H 10/60; G16H 20/00

(57) ABSTRACT

Various processes, algorithms, and systems are provided herein for assisting individuals, patients, and medical personal in evaluating the stenosis of the arteries of the heart. Methods for generating such processes, algorithms, and systems are also disclosed. In some embodiments, the method comprises receiving a set of measurements from a patient, obtaining a percentage of arterial stenosis via an analytical model, proving an alert to a physician based on the percentage of stenosis, and providing a behavioral recommendation to a patient based on the percentage of stenosis. Other aspects, embodiments, and features are also claimed and described.

### CROSS-REFERENCE TO RELATED APPLICATION(S)

This application claims the benefit of U.S. Provisional Patent Application Ser. No. 63/567,973, filed Mar. 21, 2024, the disclosure of which is hereby incorporated by reference in its entirety, including all figures, tables, and drawings.

### STATEMENT REGARDING FEDERALLY SPONSORED RESEARCH

[N/A]

### BACKGROUND

Cardiovascular disease is one of the leading causes of death in the United States.

Coronary artery disease (CAD) is the most common heart disease and is caused by the buildup of plaque inside the coronary arteries, which supply oxygen rich-blood to the heart. This narrowing of the coronary arteries is called stenosis. Despite imaging tests being one indicator of a percentage of stenosis of the arteries in the heart, imaging data alone fails to account how an individual's lifestyle, physical measurements and clinical indications can impact the percentage of stenosis of the heart. Furthermore, it can be hard to provide a real-time indicator of the percentage of stenosis of the arteries in the heart due to the invasiveness of the imaging scans. What are needed are systems and methods that provide real-time indications of the percentage of arteries in the heart with a high level of accuracy.

### SUMMARY

The following presents a simplified summary of one or more aspects of the present disclosure, to provide a basic understanding of such aspects. This summary is not an extensive overview of all contemplated features of the disclosure and is intended neither to identify key or critical elements of all aspects of the disclosure nor to delineate the scope of any of all aspects of the disclosure. Its purpose is to present some concepts of one or more aspects of the disclosure in a simplified form as a prelude to the more detailed description that is presented later.

In some aspects, the present disclosure can provide a method for determining a percentage of stenosis in arteries of a heart. A set of patient indicators, such as biographic indications, physical exam indications, an indication of hypertension, and cardiovascular dimensions from a patient's scan may be obtained. The set of patient indicators may be provided to a percentage stenosis analytical model, and a result comprising a likely percentage of arterial stenosis and a confidence interval obtained. An alert may be transmitted to a medical provider and a behavioral recommendation provided to the patient depending on the percentage of arterial stenosis determined.

In one aspect, the present disclosure provides a method for determining a percentage of stenosis in arteries of a heart, the method comprising receiving a set of patient indicators, the set comprising: a patient's biographic indications, a first set of patient physical exam indications, a first indication of patient hypertension, and a plurality of cardiovascular dimensions from patient scan data; providing the patient indicators to a percentage stenosis analytical model; via the stenosis analytical model, generating a first result comprising a likely percentage of arterial stenosis for the patient and a confidence interval; providing an alert to a user based on an upper bound of the confidence interval, wherein the alert to a medical provider is transmitted if the confidence interval encompasses a percentage of arterial stenosis equal to or greater than 70%, and providing a behavioral recommendation to the patient to lower the likely percentage of arterial stenosis if the likely percentage of arterial stenosis is greater than 50%.

In another aspect, the present disclosure provides a method for determining a percentage of stenosis in arteries of a heart, the method consisting of: receiving a set of patient indicators, the set consisting of: a patient's biographic indications, a first set of patient physical exam indications, a first indication of patient hypertension, and a first plurality of cardiovascular dimensions from patient scan data; providing the patient indicators to a percentage stenosis analytical model; wherein the percentage stenosis analytical model incorporates a measurement of minimal lumen diameter, an interaction between the minimal lumen diameter and a proximal reference lumen diameter, an interaction between a calculated percentage of a right coronary artery and distal reference lumen diameter, an interaction between a calculated percentage of the right coronary artery and a proximal reference lumen diameter, an age of the patient, an indication of body surface area, an interaction between age and an indication of body mass index, a distance to ostium from minimal lumen diameter, and the first indication of patient hypertension, via the stenosis analytical model, generating a first result comprising a likely percentage of arterial stenosis for the patient and a confidence interval; providing an alert to a user based on an upper bound of the confidence interval, wherein the alert to a medical provider is transmitted if the confidence interval encompasses a percentage of arterial stenosis equal to or greater than 70%, and providing a behavioral recommendation to the patient to lower the likely percentage of arterial stenosis if the likely percentage of arterial stenosis is greater than 50%.

These and other aspects of the disclosure will become more fully understood upon a review of the drawings and the detailed description, which follows. Other aspects, features, and embodiments of the present disclosure will become apparent to those skilled in the art, upon reviewing the following description of specific, example embodiments of the present disclosure in conjunction with the accompanying figures. While features of the present disclosure may be discussed relative to certain embodiments and figures below, all embodiments of the present disclosure can include one or more of the advantageous features discussed herein. In other words, while one or more embodiments may be discussed as having certain advantageous features, one or more of such features may also be used in accordance with the various embodiments of the disclosure discussed herein. Similarly, while example embodiments may be discussed below as devices, systems, or methods embodiments it should be understood that such example embodiments can be implemented in various devices, systems, and methods.

### BRIEF DESCRIPTION OF THE DRAWINGS

FIG. 1 is a flowchart illustrating an example of obtaining a result of percentage stenosis based on the outcome of an analytical model.

FIG. 2 is a flowchart illustrating an example generating an alert that a patient should obtain a new angiogram based on the outcome of an analytical model.

FIG. 3 illustrates a sequence of percentage stenosis in the arteries of the heart.

FIG. 4 shows an invasive coronary angiography using coronary computed tomography angiography (CCTA), according to some embodiments.

FIG. 5 shows a correlation matrix scatter plots of ₚS and the Continuous Risk Factors, according to some embodiments.

FIG. 6 shows a correlation matrix of ₚS and the Continuous Risk Factors, according to some embodiments.

FIG. 7 illustrates studentized residual plots (model diagnostics), according to some embodiments.

FIG. 8 presents a Q-Q plot of the model residuals for the test of normality of model residuals of the original data, according to some embodiments.

FIG. 9 presents component and residual plots for assess the linearity of the proposed analytical model, according to some embodiments.

FIG. 10 presents a normal Q-Q plot and a histogram and density plot from the Johnson Proposed Model, according to some embodiments.

FIG. 11 presents a residual plot of the Johnson Proposed Model to assess the homoscedasticity of the proposed analytical model, according to some embodiments.

FIG. 12 is a graph showing the observed versus predicted accuracy of the proposed analytical model, according to some embodiments.

FIG. 13 presents a contour plot of the effect of Age (X₁) and Body Mass Index, kg/m² (X₇) on percentage stenosis while keeping all other variables fixed, according to some embodiments.

FIG. 14 presents a contour plot showing the effect of Age (X₁) and proximal reference lumen diameter (X₁₆) on percentage stenosis while keeping all other variables fixed, according to some embodiments.

FIG. 15 presents a contour plot showing the effect of minimal lumen diameters (X₁₄) and proximal reference lumen diameter (X₁₆) on percentage stenosis while keeping all other fixed, according to some embodiments.

FIG. 16 presents a contour plot showing the effect of calculated percentage of RCA (X₁₀) and proximal reference lumen diameter (X₁₆) on percentage stenosis while keeping all other variables fixed, according to some embodiments.

FIG. 17 presents a contour plot showing the effect of calculated percentage of RCA (X₁₀) and distal reference lumen diameter (X₁₅) on percentage stenosis while keeping all other variables fixed, according to some embodiments.

FIG. 18 is a block diagram conceptually illustrating a system for the determining a percentage of arterial stenosis.

FIG. 19 presents a histogram showing the percentage of arterial stenosis in patients with coronary artery stenosis.

FIG. 20 is a graph showing the cumulative distribution function plot for percentage of arterial stenosis of the heart.

FIG. 21 is a graph showing the survival estimate for percentage of arterial stenosis of the heart.

### DETAILED DESCRIPTION

The detailed description set forth below in connection with the appended drawings is intended as a description of various configurations and is not intended to represent the only configurations in which the subject matter described herein may be practiced. The detailed description includes specific details to provide a thorough understanding of various embodiments of the present disclosure. However, it will be apparent to those skilled in the art that the various features, concepts and embodiments described herein may be implemented and practiced without these specific details. In some instances, well-known structures and components are shown in block diagram form to avoid obscuring such concepts.

### Example: Providing a Result of Percentage of Arterial Stenosis

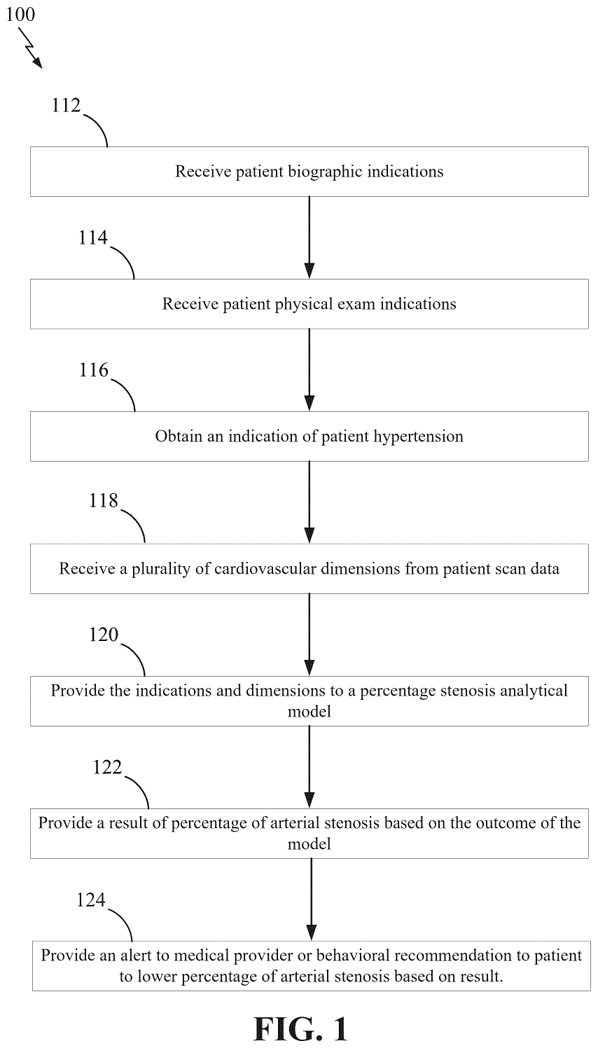

FIG. 1 is a flow diagram illustrating an example process 100 for determining a patient's percentage of arterial stenosis. In some embodiments, the process 100 can receive a set of patient indicators. In some examples, the set of patient indicators can include a patient's biographic indications, a set of physical exam indications, an indication of hypertension, and a plurality of cardiovascular dimensions from patient scan data.

In some embodiments, the process 100 can be utilized to help determine a patient's level of risk for experiencing a cardiovascular event, such as a heart attack. In other embodiments, the process 100 can be utilized to develop a patient's treatment plan to determine ways to reduce the patient's percentage of arterial stenosis, such as by suggesting a particular diet, exercise regiment, or pharmacotherapy. As described below, a particular implementation can omit some or all features/steps, may be implemented in some embodiments in a different order, and may not require some illustrated features to implement all embodiments. In some examples, an apparatus can be used to perform the example process 100. However, it should be appreciated that any suitable apparatus or means for carrying out the operations or features described below may perform the process 100.

At step 112, the process 100 can receive patient biographic indications. For example, the patient biographic indications can be indications of age (X₁) and gender (X₂). In some examples, a medical provider or assistant may input the patient biographic indications or they may be obtained from an electronic medical record, a physician database, extracted from a scan or patient chart, or may be obtained from another user or source.

At step 114, the process 100 can receive patient physical exam indications. These patient physical exam indications can include an indication of diabetes (X₄), an indication of hyperlipidemia (X₅), an indication of smoking history (X₆), an indication of body mass index (BMI; X₇), and an indication of body surface area (BSA; X₈). In some examples, a medical provider or assistant may input the patient the physical exam indications, or they may be obtained from an electronic medical record, or they may be obtained from another user or source. In some embodiments, only one of the patient physical exam indications may be used to determine the percentage of arterial stenosis result. In other embodiments, two or more of the physical exam indications can be used to determine the percentage of arterial stenosis.

At step 116, the process 100 can obtain in indication of patient hypertension (X₃). In some embodiments, the indication of hypertension can be determined from a sphygmomanometer and the value record by a user, patient, or medical professional. In other embodiments, the indication of hypertension can be determined from a wearable device, such as a watch, wrist-blood pressure cuff, or other similar device. In some embodiments, the indication of patient hypertension comprises a numerical value of 0 or 1, wherein a value of 0 indicates the patient does not have high blood pressure, and wherein a value of 1 indicates the patient does have high blood pressure. In some embodiments, a patient's high (elevated) blood pressure (hypertension) is determined based on the threshold of a systolic and diastolic blood pressure readings from the American Heart Association (AHA), standard classification. For instance, a normal blood pressure (i.e. not high blood pressure) reading indicates a systolic pressure less than 120 mmHg and a diastolic pressure less than 80 mmHg. An elevated blood pressure (prehypertension) indicates a systolic pressure between 120-129 mmHg and a diastolic pressure less than 80 mmHg. In some instances, a patient with a normal blood pressure or with prehypertension will have a value of 0 to be incorporated into the model. In other instances, a high blood pressure (stage 1) reading indicates a systolic pressure between 130-139 mmHg and a diastolic pressure between 80-89 mmHg. A high blood pressure (stage 2) reading indicates a systolic pressure of 140 mmHg or higher mmHg and a diastolic pressure of 90 mmHg or higher. In some examples, a patient with a value falling in range of stage 1, stage 2, or stage 3 will have a value of 1 incorporated into the model.

At step 118, the process 100 can receive a plurality of cardiovascular dimensions from patient scan data. In some examples, the plurality of cardiovascular dimensions can include lesion length (X₉; length of specific stenosis; measured in millimeters, or another unit of measure), fractional flow reserve (FFR; X₁₃), minimum lumen diameter (X₁₄; MILD measured in millimeters, or another unit of measure), distal reference lumen diameter (X₁₅; DRLD measured in millimeters, or another unit of measure), proximal reference lumen diameter (X₁₆; measured in millimeters, or another unit of measure), maximal lumen diameter within left main coronary artery segment (X₁₇; D_{L}M), distance between the ostium to the narrowest side (X₁₈; distance to OS from MLD).

In some embodiments, only one of the plurality of cardiovascular dimensions may be used to determine the percentage of arterial stenosis (e.g., minimal lumen diameter). In other embodiments, two or more of the plurality of cardiovascular dimensions can be used to determine the percentage of arterial stenosis. In some examples, a medical provider or assistant may input the patient the plurality of cardiovascular dimensions, or they may be obtained from an electronic medical record, or they may be obtained from another user or source. For example, the plurality of cardiovascular dimensions may be extracted from a medical image obtained from a radiology or medical imaging device. In some such circumstances, the medical device itself may have software installed thereon which automatically determines certain cardiovascular dimensions (e.g., using a segmentation or computer vision approach, and/or a neural network such as a CNN, to identify key structures and compute distances. In other circumstances, software may be provided on a remote (off-device) computer that allows a user to view images and tag measurements of the plurality of cardiovascular dimensions. In some examples, a medical provider or assistant may input the patient biographic indications or they may be obtained from an electronic medical record, or it may be obtained from another user or source.

At step 120, the process 100 can provide the indications and dimensions to a percentage stenosis analytical model that was generated based on analysis of patient records. In some embodiments, the model will determine calculations based on the indications and dimensions. For instance, the model may provide information regarding the calculated percentage of right coronary artery (RCA; X₁₀), calculated percentage of left circumflex artery (LCX, X₁₁) calculated percentage of left anterior descending artery (LAD; X₁₂). In some embodiments, DX is the maximal lumen diameter within 10-mm segment from ostium to proximal LCX, DL is the maximum lumen diameter within 10-mm segment from ostium to proximal LAD, and DR is the maximal lumen diameter within 10-mm segment from ostium to proximal RCA. In some embodiments, the calculated percentage of right coronary after can be determined by the equation: 106.1×DR/(DL+DX+DR)−9.02. In some embodiments, the calculated percentage of left circumflex artery can be determined by the equation: 140.9×DX/(DL+DX+DR)−18.24. In other embodiments, the calculated percentage of left anterior descending artery can be determined by the equation: 100−calculated percentage right coronary artery−calculated percentage left circumflex artery.

In some embodiments, the model may conceptually determine percentage stenosis by an algorithm such as:

$${p}_{} {S}_{T} = 3.8921 - 0.4299 {X}_{1}^{′} - 2.1658 {e}^{- 02} {X}_{3} + 0.1102 {X}_{8} - 3.9886 {X}_{14} - 4.9892 {e}^{- 02} {X}_{18}^{′} - 2.1255 {e}^{- 03} {X}_{1}^{′} * {X}_{7} + 0.1649 {X}_{1}^{′} * {X}_{16} + 0.6529 {X}_{10} * {X}_{15} - 0.6194 {X}_{16} * {X}_{10} + 0.3572 {X}_{14} * {X}_{16} .$$

wherein a subset of the indication and dimensions used in the model are derived from a log transformation of the original values. In some embodiments, the model may be configured to receive as inputs a specific category of input types, which each may have a range and format of acceptable values. For example, the model may have 9 inputs. In some embodiments, the user does not need to provide the list of interactions between the inputs as the software running the model calculates the interactions for certain inputs. For instance, in some examples, the model includes an interaction between minimal lumen diameter and proximal reference lumen diameter. In other examples, the model includes an interaction between the percentage of the right coronary artery and distal reference lumen diameter. In additional examples, the model incorporates an interaction between the percentage of the right coronary artery and the proximal reference lumen diameter. In further examples, the model incorporates an interaction between age and proximal reference lumen diameter. In other examples, the model incorporates an interaction between age and body mass index.

At step 122, the process 100 can provide a result of the percentage of arterial stenosis based on the outcome of the model. In some embodiments, the result could display automatically in a physician portal in an electronic medical record (EMR) system. In other embodiments, the result could display on a user interface. In some examples, this result may be obtained on a user's home computer, mobile device, or other personal device. In other examples, this result may be generated on a user's device or other system remote from the EMR, and then transmitted to an electronic medical record or clinician.

At step 124, the process 100 can utilize the output of the model from step 122 to determine an appropriate action to take (if any). For example, process 100 could provide an alert to a medical provider if the if percentage of arterial stenosis is greater than 70%. In some examples, this alert can be transmitted to an electronic medical record, or to a physician database, or to a patient.

In other examples, the process 100 can provide a behavioral recommendation to the patient to lower the percentage of arterial stenosis based on the result. In some embodiments, the process can provide a behavioral recommendation to the patient to lower the likely percentage of arterial stenosis if the likely percentage of arterials stenosis is greater than 50%. In other instances, if the percentage of arterial stenosis is over 50%, the behavior recommendations may include to improve the patient's diet by eating more fruits and vegetables, engage in additional exercise, or recommend a patient discuss pharmacotherapy to reduce one's risk of a heart attack. In other examples, the behavioral recommendation may provide a list of healthy recipes to encourage healthy eating. In further examples, the behavioral recommendation may recommend a person discuss a recommended exercise plan with the patient's medical provider. In other examples, the behavioral recommendation can provide information regarding that if a patient reduces the body mass index by a certain percentage, the patient will no longer be at a significant risk for a cardiac event. In other examples, the behavioral recommendation will provide a warning of significant health effects if the patient's body mass index continues to increase.

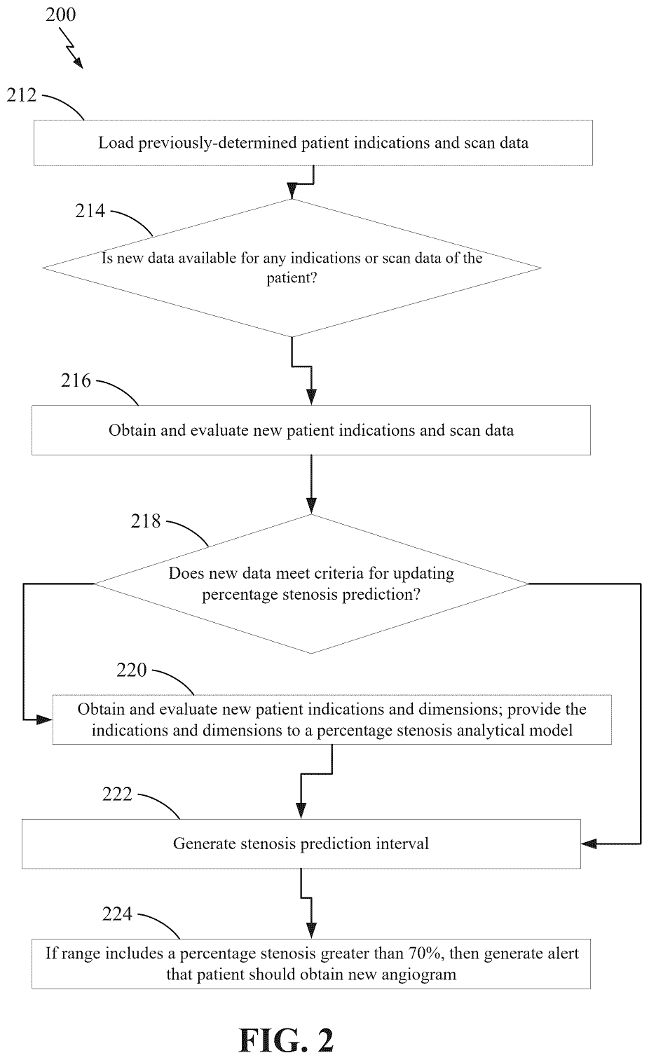

Referring now to FIG. 2, a flow diagram is shown for an example process 200 to re-evaluate the percentage of arterial stenosis of a given patient in response to changed, controllable, risk factor indicators. For example, when a patient has adopted one or more behavioral recommendations or if a change or new test/scan has occurred for any patient indicators (biographic, physical exam, hypertension) or scan data, process 200 may re-evaluate the patient's arterial stenosis. For example, in some cases process 200 may be employed in conjunction with and/or after a period of time following process 100, such as to re-evaluate a patient's percentage of arterial stenosis following a behavioral modification.

At step 212, the process 200 loads the previously-determined patient indications and scan data. For example, an initial set of data may have been previously obtained such as in relation to a process of FIG. 1. In some embodiments, this may involve accessing previously-stored data in the patient's electronic medical record, a user interface, or a physician database.

At step 214, the process 200 determines if new data is available for any risk factor indicators, or other values previously determined as patient indications or scan data of the patient. For example, process 200 may periodically monitor the electronic medical record of a given patient to determine whether a new weight/height/BMI, cardio scan, etc. has been entered since the last time a percentage stenosis was determined. In other circumstances, a physician portal may allow a physician to input information into a clinical decision support tool, to update some or all values of data previously obtained. In other embodiments, a patient or other user may be permitted to enter updated information for a given set of risk factors into a patient portal, such as weight/height, BMI, or other risk factors that (i) are predefined as those alterable by the patient; or (ii) predefined by the patient's healthcare team or software provider.

At step 216, the process 200 obtains and evaluates the new data (e.g., new values of patient indications and/or scan data). For example, where a patient inputs a new BMI determination having more/fewer decimal points, a weight measurement in a different set of units or degree of accuracy, etc., process 200 may evaluate whether the data can be normalized to a common unit of measurement/degree of precision, etc. as the original data used to generate the original stenosis percentage. For example, in some embodiments, 3D scan data may be normalized by converting to a 2D scan of a standard perspective (e.g., cross-sectional) and common scale format, so that measurements taken from the scan can be reliably translated into updated scan data. In other embodiments, where measurement information is provided, but no metadata confirming standardization of how the measurement was taken (to confirm it was equitable to the original scan information), process 200 may alert a user and request confirmation and/or may reject the data. In further embodiments, where a new scan is available (whether uploaded by a physician, scraped from an EMR, etc.), process 200 may assess whether the scan modality, image views, anatomy imaged, resolution, etc. are suitable for creating new relevant, equitable cardiovascular measurements from the scan (e.g., even if the scan was not intended for measurements of heart imaging for the specific purpose of updating a stenosis percentage).

At step 218, the process 200 evaluates if the new data meets the criteria for updating the percentage stenosis prediction. Such criteria may include factors such as: duration of time since the last update to patient indications and scan data of higher/high/similar predictive power; change in an indication that carries a correlation or relationship with another indication(s) (e.g., a significant change in BMI may correlate with a change in body surface area); potential for updated data to be inaccurate based on difference from prior measurements or population norms; and requirements implemented by a healthcare provider. For instance, if the length of time is more than 4 months between a collection of new data and original data, the process may recommend the new data may be used in recalculating the likely percentage of arterial stenosis via the stenosis analytical model. In other instances, if the patient has an increased age or has not received a measurement of minimal lumen diameter within 2 years, the process may recommend obtaining an updated scan or recalculating the percentage of arterial stenosis based on the updated age. In other embodiments, if the body mass index of a patient has changed significantly (e.g. 5%), the process may recommend or require the patient's body surface area or other related patient indications also be reevaluated. In some embodiments, if the patient's body surface area has changed by 5% or more, the most recent indication of body surface area will be used in recalculating the likely percentage of arterial stenosis via the analytical model, and providing a second result to the patient. In other instances, if the physical exam indications are significantly worse, whereby the indications are changed by at least 10% since the previous measurement, the process may recommend recalculating the percentage of arterial stenosis. In other instances, the process can determine if new data available for physical exam indications, or an indication of patient hypertension would result in a greater than 7% change of arterial stenosis (either an increase or decrease in the percentage of arterial stenosis). In some examples, if the process determines new data incorporated into the model would suggest a 7% change of arterial stenosis, the process proceeds to step 220. In other instances, if the process determines the new data available indicates the change in arterial stenosis is less than 7%, the process will proceed to step 222.

In some embodiments, the process 200 determines the new data meets criteria for updating the percentage of stenosis prediction. In these cases, at step 220 of process 200, the process will re-determine the percentage of arterial stenosis prediction using the percentage stenosis analytical model, based on the acceptable new data.

In some embodiments, the process 200 may determine the new data does not meet the criteria for updating the percentage of stenosis prediction. In some examples, the process 200 may determine the changes between the data entries are too significant a change within a duration of time that the measurements may not be accurate.

In some embodiments, the process 200 may request additional information that might be needed to perform the calculation or recommend additional testing be performed to ensure measurement accuracy. For example, where related indications must also be updated, or clinical requirements are set, the process 200 may first solicit this information form a user, patient, clinician, etc. before re-evaluating percentage stenosis.

If the data does meet the criteria for updating the percentage of stenosis prediction, or required supplemental data is given, then at step 222 the process 200 generates an updated stenosis prediction interval (confidence intervals). In some embodiments, the stenosis prediction interval is determined by:

$$CI = \hat{y} \pm {t}_{α / 2 , df} \cdot SE ( \hat{y} )$$

wherein t_{a/2,df} is the critical value from the t-distribution for a 95% confidence interval with degrees of freedom df, and
SE(ŷ) is the standard error of the predicted response,
   wherein the standard error of the predicted response is

$$SE ( \hat{y} ) = \sqrt{{{x}^{T} ( {X}^{T} X )}^{- 1} x \cdot {σ}^{2}} .$$

In some embodiments, at step 222 the process 200 generates a prediction interval. In some embodiments, the prediction interval is determined by:

$$PI = \hat{y} \pm {t}_{α / 2 , df} \cdot \sqrt{{SE}^{2} ( \hat{y} ) + {σ}^{2}}$$

where σ² is the estimated variance of the residuals.

At step 224, the process 200 evaluates if the range includes a percentage stenosis greater than 70%, then an alert is transmitted to an electronic medical record or physician database indicating the patient should obtain a new angiogram.

### Example: Hardware Integration of Data-Driven Analytical Model

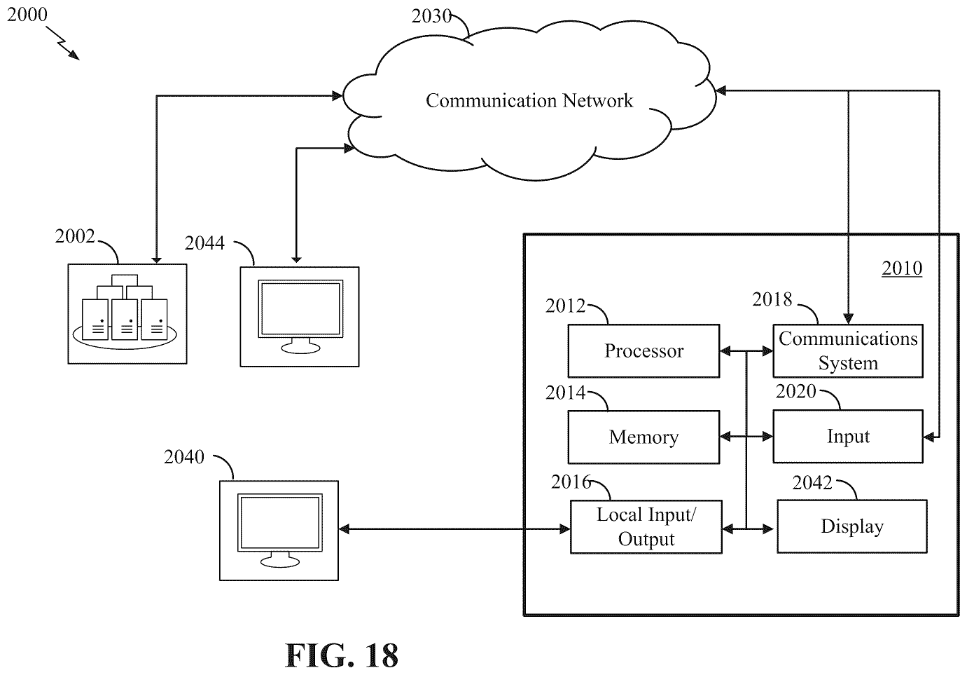

FIG. 18 shows a block diagram illustrating a system 2000 for the determining the percentage of arterial stenosis described herein, using a non-transitory computer readable medium according to some embodiments. In one respect, the process can be thought of as a way of verifying or communicating the results of a percentage of arterial stenosis using the analytical model. In other aspects, the process may provide a recommended behavior modification based on the determination of the percentage of arterial stenosis. As shown, the computing device 2010 can be an integrated circuit (IC), a processor, server, cloud resource, or any suitable computing resource. Thus, the processes 100 and 200 described in FIGS. 1 and 2 can be implemented for or by the computing device 2010.

In the system 2000, a computing device 2010 includes a data communications link such that it can obtain or receive a dataset. The dataset can be a set of indications and scan data found in the electronic medical record 2002, or any other suitable dataset for running processes such as process 100. For example, the dataset can include data obtained from extracted patient records, patient self entry, medical provider self-entry, or a preexisting dataset. In other examples, one or more features can be extracted from the dataset and then only the relevant features can be applied to the non-transitory computer readable medium. The computing device 2010 can receive the dataset, which is stored in a database, via communication network 2030 and a communications system 2018 or an input 2020 of the computing device 2010.

The computing device 2010 can include an electronic processor 2012 and a memory 2014. The memory 2014 can include any suitable storage device or devices that can be used to store suitable data (e.g., a software application running a user interface, an integration to an electronic medical record, etc.) and software instructions that can be used, for example, by the processor 2012. The memory 2014 can include a non-transitory computer-readable medium including any suitable volatile memory, non-volatile memory, storage, or any suitable combination thereof. For example, memory 2014 can include random access memory (RAM), read-only memory (ROM), electronically-erasable programmable read-only memory (EEPROM), one or more flash drives, one or more hard disks, one or more solid state drives, one or more optical drives, etc., or may simply be an apportioned cloud, network, or other resource. In some embodiments, the processor 2012 can execute at least a portion of processes 100 and 200 described above in connection with FIGS. 1 and 2.

The computing device 2010 can further include a communications system 2018. The communications system 2018 can include any suitable hardware, firmware, and/or software for communicating information over the communication network 2030 and/or any other suitable communication networks. For example, the communications system 2018 can include one or more transceivers, one or more communication chips and/or chip sets, etc. In a more particular example, the communications system 2018 can include hardware, firmware and/or software that can be used to establish a Wi-Fi connection, a Bluetooth connection, a cellular connection, an Ethernet connection, etc.

The computing device 2010 can receive or transmit information (e.g., electronic medical record 2002, a physician database 2044 etc.) and/or any other suitable system over a communication network 2030. In some examples, the communication network 2030 can be any suitable communication network or combination of communication networks. For example, the communication network 2030 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard, such as CDMA, GSM, LTE, LTE Advanced, NR, etc.), a wired network, etc. In some embodiments, communication network 2030 can be a local area network, a wide area network, a public network (e.g., the Internet), a private or semi-private network (e.g., a corporate or university intranet), any other suitable type of network, or any suitable combination of networks. Communications links shown in FIG. 18 can each be any suitable communications link or combination of communications links, such as wired links, fiber optic links, Wi-Fi links, Bluetooth links, cellular links, etc.

In some examples, the computing device 2010 can further transmit an output connection 2016 to a user interface 2040. The output 2016 connection may be part of or rely upon a network connection such as the communication link 2030, but alternatively may be a separate connection such as, e.g., a private connection to a healthcare organization's electronic medical record system or may include other connections such as an email server. The form of output connection 2016 may depend upon the form of data to be provided to a user as well as where the computing device 2010 resides. As another example, if the computing device 2010 is hosted by a healthcare organization or clinic, the output may comprise all or a portion of a user interface directed to the treating provider. In some embodiments, the output connection 2016 can transmit percentage of arterial stenosis, a behavioral recommendation, a provider alert, a patient alert, and/or other information. In other examples, the output 2016 can include a display to output a prediction range of stenosis. In some embodiments, the display 2042 can include any suitable display devices, such as a computer monitor, a touchscreen, a television, an infotainment screen, etc. to display the result or behavioral recommendation. In further examples, the result, behavioral recommendation or any other information pertaining to the percentage stenosis analytical model can be transmitted to another system or device over the communication network 2030.

In further examples, the computing device 2010 can include an input connection 2020. The input connection 2020 can be coupled to a communication link such as network 2030 for receipt of data from remote locations (e.g., patient indications and scan data, etc.) or may be an integration to a locally-controlled electronic medical record or other healthcare software. For example, the input connection 2020 may receive a set of patient indications and scan data corresponding to the electronic medical record 2002. In other examples, the input 2020 can include any suitable input devices (e.g., a keyboard, a mouse, a touchscreen, a microphone, etc.) and/or the one or more sensors that can produce the raw sensor data or the dataset 2002.

### Example: Phase I—A Real Data-Driven Analytical Predictive Model of the Percentage Stenosis of the Artery of the Heart (PSAH)

### Overview

This phase of the innovation is to develop a real data-driven analytical predictive model for the percentage of arterial stenosis in patients with coronary artery stenosis. The developed model conveys useful information about a patient's percentage stenosis in the arteries of the heart, PSAH. It identifies the risk factors, individually and interactively, that drive the PSAH of patients likely to experience cardiovascular events such as heart attacks or strokes with 96% accuracy. More specifically, the proposed analytical real data-driven model offers useful findings of what causes the percentage stenosis of the arteries of the heart that results in a heart attack or stroke in a given patient with 96% accuracy. Firstly, it identifies the individual risk factors that statistically significantly contribute to the percentage of arterial stenosis in patients. Secondly, it identifies the interaction of the risk factors that statistically significantly contribute to the percentage of arterial stenosis. Thirdly, the inventors obtain the ranks of the individual and interaction risk factors according to the percentage contribution to the percentage of arterial stenosis, from the largest to the smallest contribution. Lastly, given the information on the risk factors of a given patient, the developed model predicts with at least a 96% accuracy the percentage of arterial stenosis. Finally, the developed analytical model will be used in Phase II to develop an optimization method that identifies the risk factors that will minimise the percentage stenosis in the arteries of the heart, PSAH.

### Introduction

The function of the heart is to pump the blood that bathes and nourishes every organ of the body. The blood carries the oxygen and nutrients vital to the tissues, and it also carries waste products away from the tissues. If the pumping action of the heart is disrupted for any reason, the body's organs begin to fail very quickly. So, life itself is dependent on the efficient, continuous operation of the heart.

The human heart is roughly the size of a fist and the weighs about 300 grams and beats 60-80 times per minute throughout your entire life. It pumps 5-6 Liters of blood throughout the body. Systole and diastole process in the heart helps in the blood circulation. The period of contraction (systole) in the heart chambers must alternate with the period of relaxation (diastole) in order for the heart to function properly. During systole, a contraction chamber will eject blood, and during diastole, a relaxed chamber will fill with blood. Incomplete filling or ejection can lead to inadequate pumping of the blood to tissues.

Heart disease, also known as cardiovascular disease, refers to a group of conditions that affect the heart and blood vessels. It is a broad term that encompasses various disorders and conditions related to the heart and its functioning. Coronary Artery Disease (CAD) is the most common type of heart disease and is caused by the buildup of plaque inside the coronary arteries, which supply oxygen-rich blood to the heart muscle (Myocardium). This can lead to a reduced blood flow and potentially cause chest pain or a heart attack (Myocardial Infarction).

A heart attack occurs when there is a sudden blockage in one or more of the coronary arteries, leading to a loss of blood supply to a part of the heart muscle. This can result in damage to the heart tissue and the heart's unable to pump blood effectively, causing a backlog of blood in the heart or lungs. The most common disease caused by stenosis is coronary artery disease. It's the leading cause of death worldwide, affecting millions of people and accounting for around 60-70% of all cases of arterial disease.

Coronary artery disease is a significant health concern in the United States, with an estimated 18.2 million adults in 2019 had been diagnosed with coronary heart disease. The prevalence increases with age, affecting both men and women. CAD remains a leading cause of death not only in the United States but also globally, affecting over 120 million people.

Coronary artery disease is the leading cause of death for men, women, and people of most racial and ethnic groups in the United States. According to the CDC, heart disease was responsible for approximately 1 in every 5 deaths, and one person dies every 33 seconds from cardiovascular disease in the United States in 2021.

Research continues to develop medications to manage the subject disease, as the well as advances in interventional cardiology have led to procedures like angioplasty and stent placement to restore blood flow in blocked arteries and alleviate symptoms of CAD.

### Problem Statement

The narrowing of the coronary arteries can be gradual over time, and it's often caused by the accumulation of cholesterol, fatty deposits, calcium, and other substances on the inner walls of the arteries. There are two main coronary arteries: the left and right coronary arteries. Over time, these arteries can become atherosclerotic, which is the formation of fatty plaques on the walls. Atherosclerotic plaques have a fibrous cap that contains thrombogenic material (cell debris, lipids, and inflammatory cells). In some cases, these plaques are developed over the years and extend far enough into the lumen of the vessels to narrow it, which can lead to ischemia.

The coronary arteries are end-arteries; that is, they supply blood to a discrete area of the myocardium and have limited collateral circulation. End-arteries are also susceptible to obstruction by atherosclerotic plaque or thrombus that can result in loss of blood flow to the myocardial muscle normally supplied by that artery. This can be fatal, depending on the location of the obstruction. Blockage of coronary arterial blood flow, especially in the left main coronary artery, usually results in death from massive infarction of the left ventricle. If the blocked artery supplies a smaller section of the myocardium, the result may be a myocardial infarction but not death. If any of the coronary arteries get blocked, it results in a heart attack because the myocardium isn't getting the oxygenated blood required for its function, and the muscle begins to die.

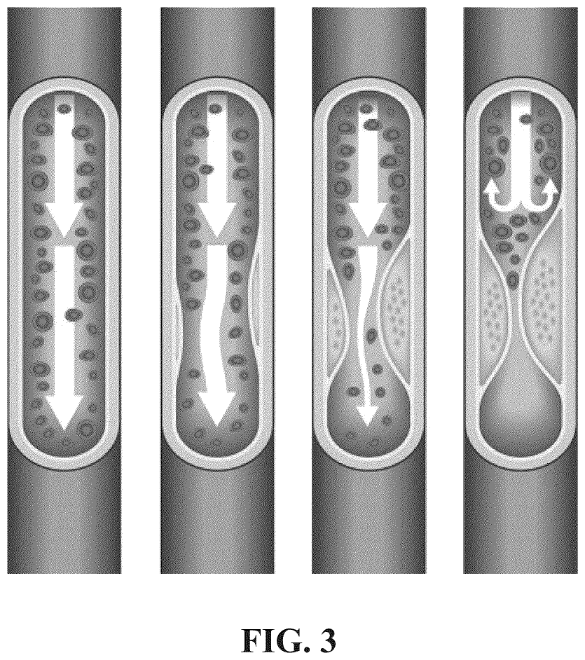

End arteries supply the myocardium without sufficient overlap or anastomoses from other coronary arteries. Since each artery is an end artery, if there is a blockage (X) in an artery, the rest of the tissue downstream no longer gets any oxygenated blood. One would think that looking at the connections of the arteries, it would be an anastomoses connection or overlapping so that blood would be able to go retrogradely to supply the other area, but it was found from clinical studies that this does not occur and the whole area downstream suffers from ischemic damage or heart attack, or cardiac arrest, or myocardial infarction. The myocardium has to have oxygen, and because each of its vessels is an end artery, if it gets blocked, the heart suffers fatally. The sequence of Percentage stenosis in the arteries of the heart is shown in FIG. 3.

### Data Description

The data for the study was obtained from the PLOS Medicine Open data repository, a biomedical research cooperation in San Francisco, California, consisting of 1,132 stable and unstable angina patients who underwent invasive coronary angiography using coronary computed tomography angiography (CCTA) and/or functionally confirmed one vessel coronary artery disease, percentage diameter stenosis by quantitative coronary analysis, QCA.

It consists of 18 risk factors divided into the patient's clinical and angiographic characteristics. The clinical risk factors are further divided into modifiable and non-modifiable risk factors. Modifiable risk factors include smoking, hypertension, diabetes, hyper-lipidemia, BMI, and BSA. The non-modifiable risk factors include age, sex, family history, etc. The variable of interest under study is the percentage stenosis of the artery.

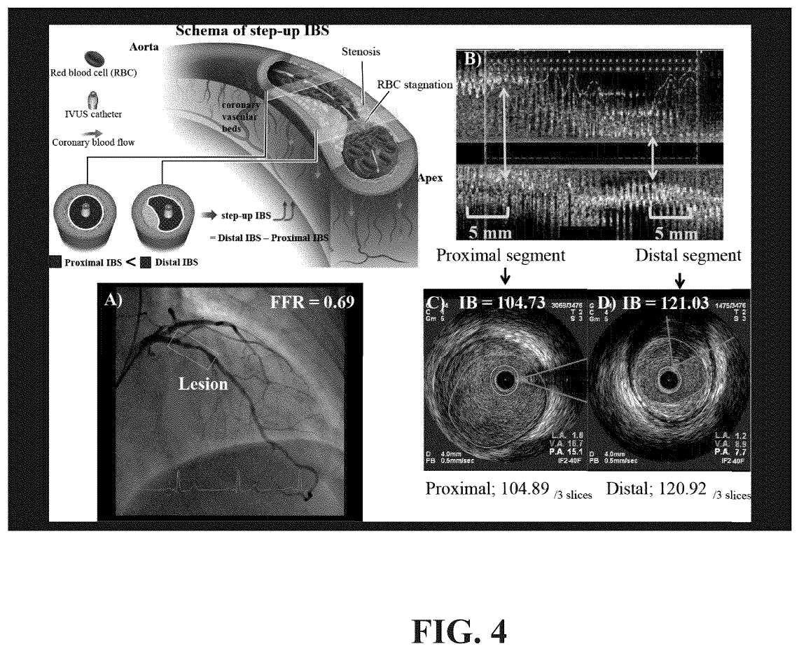

The invasive coronary angiography using CCTA is shown in FIG. 4.

Example definitions of the variables (risk factors) in the data are as follows:

In some embodiments, lesion length refers to the length of a specific narrowing or blockage in a blood vessel. It's used most commonly in the context of coronary artery disease, and it can help doctors determine the severity of the condition and the best treatment options.

Lumen refers to the hollow space or cavity inside the blood vessel or artery. The size and shape of the lumen can impact the flow rate and pressure of the fluid passing through it and the Lumen diameter is the measurement of the width of that hollow space cavity.

Minimal lumen diameter is the measure of the narrowest point within a blood vessel like the artery.

Proximal minimal lumen diameter refers to the narrowest point of the lumen that's closest to the point of origin of a blood vessel or artery.

Distal minimal lumen diameter refers to the narrowest point of the lumen that's furthest away from the point of origin of the blood vessel.

Ostium is the opening of a blood vessel that connects that tubular structure to another tubular structure or cavity. A coronary artery has an ostia that connects them to the aorta.

Myocardial volume refers to the amount of blood that the heart muscle can pump in a given amount of time, which can provide important information about the overall health and function of the heart. A higher myocardial volume is associated with a stronger, healthier heart, while a lower volume can indicate heart muscle weakness or damage.

Fractional flow reserve (FFR) is a measurement used to assess the severity of coronary artery stenosis (narrowing) in the heart. It measures the pressure difference between the distal (downstream) and proximal (upstream) parts of a coronary artery. If the FFR is low (below 0.75), it indicates that the stenosis is significant and could be causing symptoms of ischemia (reduced blood flow to the heart muscle).

Further Abbreviations of the data: LAD: left anterior descending artery; LCX: left circumflex artery; RCA: right coronary artery. By angiography, calculated % RCA, calculated % LCX, and calculated % LAD were estimated as follows using the current model as attributes.

$$calculated \% RCA = 106.1 \times DR / ( DL + DX + DR ) - 9.02$$

$$calculated \% LCX = 140.9 \times DX / ( DL + DX + DR ) - 18.24$$

$$calculated \% LAD = 100 - calculated \% RCA - calculated \% LCX$$

Table 1 is the dictionary of clinical characteristics and angiographic features of the data.

TABLE 1
Variables: Clinical and Angiographic Characteristics
of Patient with Coronary Artery Stenosis.

| | | |
|---|---|---|
| Variable Name | Symbol | Variable Discription |

Response Variable

| | | |
|---|---|---|
| Percentage | ₚS | Diameter stenosis of the |
| Stenosis | | stenotic segment |

Clinical Characteristics

| | | |
|---|---|---|
| Age | X₁ | Age of patient at diagnosis |
| Gender | X₂ | Sex of patient |
| Hypertension | X₃ | Does patient have high blood |
| | | pressure (0 = No. 1 = Yes) |
| Diabetes Mellitus | X₄ | Does patient have diabetes |
| | | (0 = No. 1 = Yes) |
| Hyperlipidemia | X₅ | Does patient have high lipids, |
| | | cholesterol triglycerides |
| | | (0 = No. 1 = Yes) |
| Smoking | X₆ | Current smoking status of |
| | | patient (0 = No. 1 = Yes) |
| BMI | X₇ | Body mass index, kg/m² |
| BSA | X₈ | Body surface area, m² |

Angiographic Features

| | | |
|---|---|---|
| Lesion length | X₉ | Length of specific stenosis, mm |
| Calculated % RCA | X₁₀ | Estimated percent myocardial |
| | | volume supplied by the RCA |
| Calculated % LCX | X₁₁ | Estimated percent myocardial |
| | | volume supplied by the LCX |
| Calculated % LAD | X₁₂ | Estimated percent myocardial |
| | | volume supplied by the LAD |
| FFR | X₁₃ | Fractional flow reserve |
| MLD | X₁₄ | Minimal lumen diameter, mm |
| Distal RLD | X₁₅ | Distal reference lumen |
| | | diameter, mm |
| Proximal | X₁₆ | Proximal reference lumen |
| RLD, mm | | diameter, mm |
| D_{L}M | X₁₇ | Maximal lumen diameter within |
| | | left main coronary artery segment |
| Distance to OS | X₁₈ | Distance between the ostium to |
| from MLD | | the narrowest site |

DX: Maximal lumen diameter within 10-mm segment from ostium to proximal LCX.
DL: Maximal lumen diameter within 10-mm segment from ostium to proximal LAD.
DR: Maximal lumen diameter within 10-mm segment from ostium to proximal RCA.

### The Art of Developing an Analytical Predictive Model for the Percentage Stenosis in the Artery of the Heart

The objective of this section is to develop an analytical predictive model based on real data to identify significant attributable risk factors affecting the percentage of arterial stenosis in patients with coronary artery stenosis, with a high degree of accuracy. The dataset comprises 1,132 patients with stable and unstable angina who underwent invasive coronary angiography, utilizing coronary computed tomography angiography (CCTA) and/or functionally confirmed one-vessel coronary artery disease (percentage diameter stenosis by quantitative coronary analysis, QCA). It encompasses 18 risk factors categorized into the patient's clinical and angiographic characteristics.

To ensure the fulfillment of all analytical modeling assumptions, the inventors meticulously filtered the data. Following the development of statistical modeling, the inventors pinpointed the significant individual attributable variables or risk factors, along with interaction terms contributing to the percentage of arterial stenosis in patients. These factors, including interaction terms, were then ranked based on their percentage contribution to arterial stenosis in patients.

The proposed model's quality and accuracy the were assessed using various statistical measures, such as R², R²_{adjusted} statistic, Akaike information criterion (AIC) for model selection, prediction error sum of squares (PRESS), root mean square error (RMSE), variance inflation factor (VIF), residual analysis, and prediction accuracy (correlation of actual and predicted percentage stenosis in the artery based on 80% training set and 20% testing data sets).

The insights provided by the proposed model offer medical professionals crucial information on the degree and severity of stenosis in the arteries of the patient's heart, guiding appropriate treatment measures or options.

### Analytical Modeling

Through the art of developing a statistical model for multivariate linear regression, the following assumptions must be satisfied:

1) Linearity: The risk factors should have a linear relationship with the response variable (Percentage Stenosis). This can be expressed as (Equation 1):

$${p}_{} {S}_{i} = η + {\sum }_{i = 1}^{p} {γ}_{i} {X}_{i} + {\sum }_{i \neq j = 1}^{p} {ψ}_{ij} {X}_{i} {X}_{j} + {ϵ}_{i}$$

where the response variable ₚSᵢ,=(ₚS₁, . . . , ₚ Sₙ)^{T}, η=(1, . . . 1)^{T} is the intercept or constant term, γᵢ=(γ₁, . . . , γₖ)^{T} is the coefficient parameter of the attributable factors Xᵢ's, ψᵢⱼ, is the coefficient parameter of interaction between i^{th} and j^{t}h risk factors, ∈ᵢ=(∈₁, . . . , ∈ₙ)^{T} denotes the model residual error term, p=18 is the number of risk factors given in Table 1, and n=1, 132 is the sample size of the data. Linearity was assessed using the correlation matrix between the response and the continuous explanatory variables.

2) Normality: Ensuring the accuracy of our model, it is essential to assess the normality of the model residuals. Specifically, the inventors assess whether the errors follow a Gaussian normal probability distribution with zero mean and standard deviation of one, denoted by ϵ˜N(0, 1) as n→0. A visual assessment of normality was conducted using a normal probability Q−Q plot. Additionally, formal tests of normality, including Shapiro-Wilk's test and Anderson-Darling, the were employed, with the null hypothesis H₀ stating that the residual errors follow the normal probability distribution. The outcomes of these tests were utilized to affirm the normality assumption of errors in our model.

3) Homoscedasticity is a critical assumption in linear regression models, asserting that the variance of residuals should remain constant across all levels of the independent variables. To validate this assumption, the inventors initially examine the plot of residuals against fitted values. The absence of discernible patterns in the plot suggests constant variance of errors, denoted as var(ϵᵢ)=a². Subsequently, the inventors conduct a formal test for non-constant variance with the null hypothesis H0 stating that the variance of errors is constant.

4) Zero or minimal multicollinearity: To avoid unstable estimates of model coefficients, the risk factors should not exhibit multicollinearity. Typically, a correlation coefficient of r≥0.9 indicates substantial correlation. A formal test for multicollinearity involves employing the variance inflation factor (VIF), calculated as

$$VIF = \frac{1}{1 - {R}_{\text{?}}^{\text{?}}} \text{?}$$

$$\text{?} \text{indicates text missing or illegible when filed}$$

a VIF>10 implies the existence of multicollinearity.

5) No autocorrelation: The residual errors should be independent and uncorrelated, denoted as ϵ˜i.i.d/N(0, σ²). To test this assumption, the Durbin-Watson test is employed with the null hypothesis H₀, asserting the absence of autocorrelation.

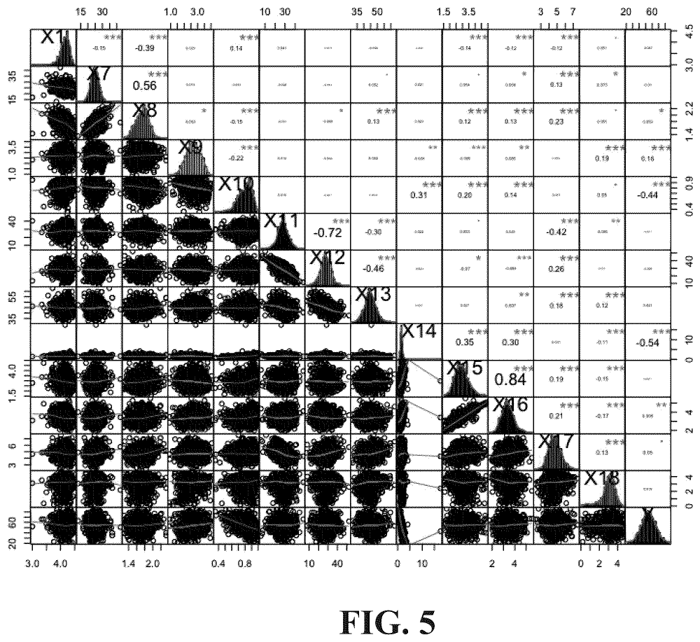

The inventors initiated the analysis by visually examining the matrix of scatter plots to evaluate the linear relationship between the response variable ₚS and the continuous risk factor X. As illustrated in FIG. 5, a weak linear relationship is observed between the response variable ₚS and almost all the continuous risk factors, with the highest correlation coefficient being r=−0.54, indicating a moderate correlation with X₁4.

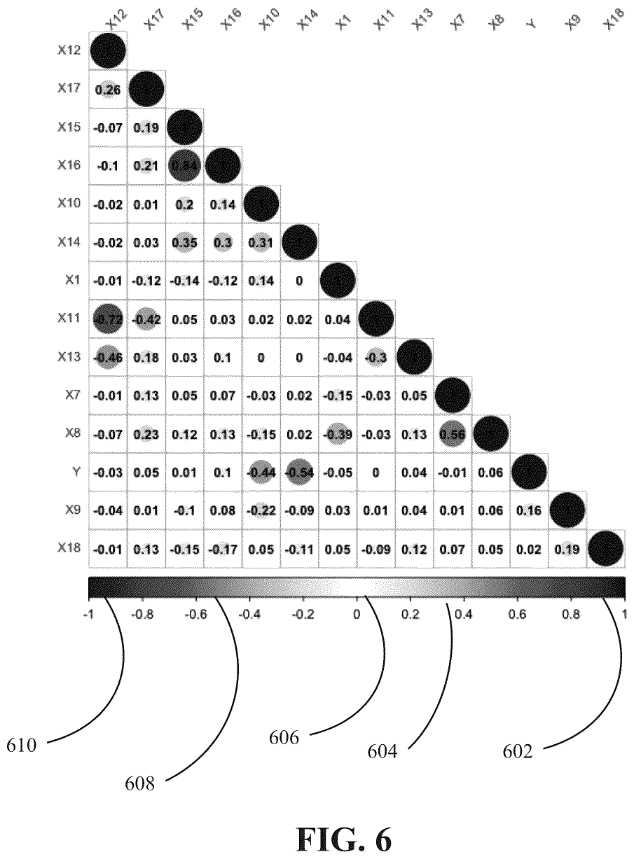

The correlation coefficients were obtained from the correlation matrix depicted in FIG. 6. These correlation coefficients measure the strength of the linear relationship between two variables, spanning a range from −1 to 1. A correlation diagram utilized depicts a visualization of the correlation between variables, where dark shading 602 represents a strong positive (+ve) correlation, light shading 604 represents a moderate positive correlation, and white (no shading) 606 represents little or no correlation. Similarly, deep brown shading 610 and light brown 608 represent strong and moderate negative (−ve) correlations, respectively. The distribution of the phenomenon of interest ₚS exhibits a slight right-skewness, as it follows the three-parameter Fatigue-Life probability distribution identified through parametric analysis.

Notably, some risk factors display skewed-shaped distributions, particularly X₁₄, hinting at a possible influence of outliers or extreme values. Although the inventors are monitoring X₁₄, no action has been taken at this point.

Continuing with the analysis, the inventors proceeded to fit the initial model of the response variable as a function of the 18 attributable risk factors, yielding a coefficient of determination R² of 0.4622, approximately 46%.

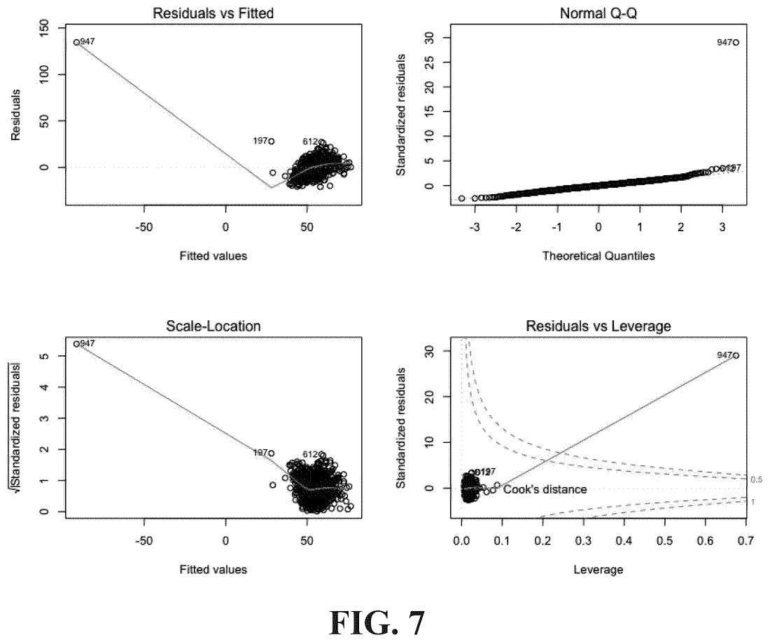

This indicates a suboptimal model, considering the low R² value. To identify issues with the model, a diagnostic was performed, which consisted of studentized residual plots and Cook's distance, revealing the presence of an outlier in all four sections of the plot (see FIG. 7).

Following the removal of this observation (947) from the data and refitting the model, the inventors achieved a higher R² of 0.9323, approximately 93%, compared to the initial model.

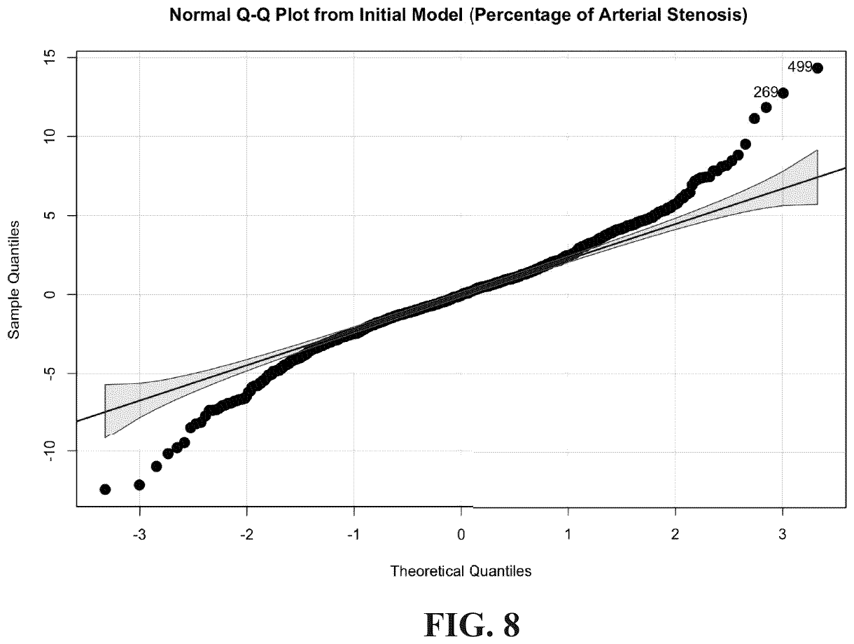

Despite achieving a high R² for our model, it is imperative to ascertain the satisfaction of all other model assumptions. To thoroughly assess these assumptions, the inventors conducted a model diagnostic. Initially, the inventors examined the assumption of normality by inspecting the Q−Q plot of the model residuals. The plot exhibited evidence of deviation from normality, with skewed ends where a few points fell outside the 95% confidence bound (see FIG. 8). This deviation was further confirmed by formal tests for normal distribution. The Shapiro-Wilk's normality test yielded a p-value of 1.744e−¹³, indicating a lack of adherence to the normal probability distribution. Additionally, an Anderson-Darling test produced a p-value of 1.021e−⁵, further supporting the violation of the normal probability distribution assumption.

The assumption of homoscedasticity of the model residuals was found to be violated, as indicated by the formal test of non-constant variance score test yielding a p-value of 4.5651e−⁷.

Another issue arose with the initial fitted model, involving an insignificant intercept-a critical concern in multivariate regression. The intercept holds substantial significance in any linear regression model, aiding in the adjustment for the linearity between the response and the predictors. An insignificant intercept suggests that when all predictors are zero, the response variable is also zero, posing a statistical fallacy. Therefore, ensuring a significant intercept is essential for the model to be statistically sound and justified.

Given the number of discrepancies encountered in our initial model, the inventors implemented several remedies to tackle the issues. The inventors checked the variance of all 18 risk factors to address the issue of unstable estimates of model coefficients. The inventors identified that variables X₁, X₉, and X₁₈ had very high variance. Log transformation stabilizes the variance and suppresses the impact of outliers or extreme values in the data. The transformations are given by the expressions below (Equation 2):

$${X}_{i}^{′} = \{ \begin{matrix}- log ( - {X}_{i} + 1 ) , & if x < 0 \\ log ( {X}_{i} ) , & if x > 0 \\ log ( {X}_{i} + 1 ) , & otherwise .\end{matrix} \qquad ( 2 )$$

Depending on the type of model and the specific assumption violation, various remedies can be used, such as transformations. Transformations, such as the Box-Cox transformation, logarithmic, polynomial, square root, inverse, and square transformations, can be used to address certain types of discrepancies.

The inventors conducted multiple transformations on the response and performed model diagnostics on each fitted model to ascertain a model that yields a high R² and can satisfy all our model assumptions. Additionally, the inventors tackle the issue of identifying and removing outliers and extreme values that impact the quality of our proposed model. To identify real influential observations that could actually have large impact on the regression result, the inventors run the Cook's Distance test. After performing the transformation, the inventors refitted the model with all 18 attributable variables, including their two-way interactions with the transformed response. It is, however, important to note that selecting the appropriate risk factors for a regression model is not a trivial task. Too few variables may lead to biased models, while too many variables may lead to overfitting and excess variability.

The inventors employed the backward stepwise elimination model selection method to select the significant contributing risk factors and interactions. This method is an efficient model selection technique that helps to prevent model over-fitting, provides less bias mean square error (MSE) values, and enhances model prediction performance. It uses the Akaike information criterion (AIC) to select the best model with the least AIC. The AIC estimates the relative amount of information loss in the model; hence, the smaller the AIC, the better the fit of the model. Following the outlined process, the inventors derived the best transformations, namely the Johnson transformation, that enabled us to achieve the high-quality proposed model with our desired results.

The proposed Johnson transform analytical model identified ten (10) significant risk factors consisting of five (5) individual factors and five (5) interaction terms contributing to the percentage stenosis of patients with coronary artery stenosis in the heart. The equation of the proposed model is given by Equation 3:

$${}_{p} {S}_{T} = 3.8921 - 0.4299 {X}_{1}^{′} - 2.1658 {e}^{- 02} {X}_{3} + 0.1102 {X}_{8} - 3.9886 {X}_{14} - 4.9892 {e}^{- 02} {X}_{18}^{′} - 2.1255 {e}^{- 03} {X}_{1}^{′} * {X}_{7} + 0.1649 {X}_{1}^{′} + 0.6529 {X}_{10} * {X}_{15} - 0.6194 {X}_{16} * {X}_{10} + 0.3572 {X}_{14} * {X}_{16} \qquad ( 3 )$$

along with the non-linear Johnson transformation form for the analytical proposed model, given by (Equation 4):

$${}_{p} {S}_{T} = γ + η ln ( \frac{{}_{p} S - ξ}{λ + ξ - {}_{p} S} ) , ξ < {}_{p} S < ξ + λ \qquad ( 4 )$$

where ₚS_{T} denotes the transformed response, ₚS is the non-transformed response, and γ, η, λ and ξ are the transformation parameters. with,

−∞<γ<∞,γ−the shape parameter,

η>0,η−the shape parameter,

λ>0,λ−the scale parameter,

−∞<ξ<∞,ξ−the location parameter,
Substituting the parameter estimates into Equation [4] gives (Equation 5):

$${}_{p} {S}_{T} = 2.1184 + 2.927 ln ( \frac{{}_{p} S - 4.3531}{4.8113 - {}_{p} S} ) . \qquad ( 5 )$$

For the purpose of accurate prediction, the inventors can't leave our response in the transformed form of the model; the inventors need to back-transform the transformed response ₚS_{T} and the transformed attributable risk factors X′i to attain the original form. In light of this, the inventors utilized the anti-logarithmic to back-transform the transformed attributable risk factors X′₁ and X′₈ to their original form with Equation [2] below:

$${X}_{i} = \{ \begin{matrix}1 - {e}^{- {X}_{\text{?}}^{′}} , & if x < 0 \\ {e}^{- {X}_{\text{?}}^{′}} , & if x > 0 \\ - 1 + {e}^{- {X}_{\text{?}}^{′}} , & otherwise .\end{matrix} \qquad ( 6 )$$

$$\text{?} \text{indicates text missing or illegible when filed}$$

Equation [7] below estimates the transformed percentage stenosis based on the Johnson transformation. By performing an anti-transform or back-transformation on Equation [5], the inventors can obtain the non-linear analytical form of the response value in its original form in Equations [7] and Equation [8](after substituting the parameter estimates). This enables us to express the percentage stenosis in its original, non-linear analytical form, which is essential for accurate prediction.

$$= ξ + \frac{λ}{1 + exp ( \frac{γ - {}_{p} {S}_{T}}{η} )} \qquad ( 7 )$$

$$and$$

$$= 4.3531 + \frac{0.4582}{1 + exp ( \frac{2.1184 - {}_{p} {S}_{T}}{2.937} )} . \qquad ( 8 )$$

Our proposed non-linear analytical model for the percentage stenosis in the arteries of the heart in patients with coronary artery stenosis is expressed by Equation [8]. This model incorporates five (5) significant individual attributable variables and five (5) significant interaction terms, resulting in a high R²=0.9616 (96.16%) and R²_{adj}=0.9612 (96.12%), indicating the development of a very high-quality model. The R² values represent the proportion of variation in the response variable ( [symbol] ), which can be explained by the relationship between the five significant individual risk factors and the five interaction terms in Equation [3]. A higher R² indicates a better goodness-of-fit of the model. The analytical form of R² and R^{2′}_{adj} is given by

$${R}^{2} = 1 - \frac{SSE}{SST} ,$$

$$and$$

$${R}_{adj}^{2} = 1 - \frac{SSE / ( n - k )}{SST / ( n - 1 )} ,$$

where: SSR=Σᵢ(ₚ\hat{ }Sᵢ−ₚ S)² is the regression sum of squares representing the variation explained by the significant attributable variables in the proposed model. SSE=Σᵢ(ₚSᵢ−ₚ\hat{ }Sᵢ)²=Σᵢ²ᵢ is the error sum of squares; and ₚ S=1/nΣⁿ_{i p}Sᵢ are the percentage stenosis, ₚ\hat{ }Sᵢ is the estimated percentage stenosis. SST=Σᵢ(ₚSᵢ−p S)², is the total sum of squares which is the proportion to the sample variance and equals to the sum of SSR and SSE. The coefficient of determination, R², has a known issue of increasing as the number of parameters or predictors in the model increases. To address this issue, it is recommended to report R² along with its adjusted version R²−adj, which takes into account the degree of freedom of the model (where R²_{adj}≤R²). The degree of freedom of SSE is denoted by n−k, and n−1 is the degree of freedom of SST. The goodness-of-fit of the model can be evaluated based on the proximity of R²−adj to R², with a smaller difference indicating a better fit and the absence of bias.

To utilize the proposed model for predicting the percentage stenosis in the arteries of the heart in patients with coronary artery stenosis or new patients seeking information about their arterial stenosis degree or severity, the inventors initially input the values of the identified significant attributable risk factors and interaction terms into the proposed model. This process yields the transformed response or percentage stenosis, ₚS_{T} Equation [5]. Subsequently, to obtain the actual value of the percentage stenosis, [symbol] ), the inventors perform an anti-transformation using Equation [8]. Through this approach, given the values of the risk factors identified as contributors to percentage stenosis in the proposed Johnson analytical predictive model, the inventors can accurately predict the percentage stenosis in the arteries of the heart of a patient with a high degree of accuracy of at least 96%.

### Ranking the Identified Significant Individual Attributable Risk Factors and the Interaction Terms

The proposed non-linear analytical model is able to rank the identified significant attributable risk factors and interaction terms based on their percentage contribution to the percentage stenosis in the arteries of the heart in patients with coronary artery stenosis.

From the presented Table [2], which displays the ranking order of each identified attributable risk factor and interaction term according to the percentage of contribution to the percentage stenosis based on the R² statistic, it is evident that Minimal Lumen Diameter (MLD) (X₁₄), a measure of the narrowest point within a blood vessel like the artery, holds the top rank. MLD provides valuable insights into the extent of narrowing or blockage in the vessel. Smaller MLD values are associated with an increased risk of future cardiovascular events, such as heart attacks or strokes.

Also, Age (X₁) can be seen interacting with two distinct risk factors and standing as a significant risk factor itself. Age is recognized as a major risk factor for atherosclerosis, the underlying process leading to stenosis. As individuals age, their arteries become less flexible and more susceptible to plaque buildup. The prevalence of atherosclerosis, and consequently stenosis, steadily increases with age.

Hypertension (X₃), ranking last (10th) among the risk factors, represents the medical term for high blood pressure. This condition occurs when the force of blood against the walls of blood vessels is higher than normal. Hypertension is a critical risk factor for cardiovascular disease, capable of causing damage to the heart and blood vessels over time. Various factors, including diet, lifestyle, certain medications, and chronic conditions, can contribute to high blood pressure.

A detailed discussion of the rankings will continue in the next section.

TABLE 2
Rank of the Significant Attributable Risk Factors
and Interaction Terms based on their percentage
of contribution to the percentage stenosis

| | | | | % |
|---|---|---|---|---|
| Rank | Symbol | Variable | R² | Contribution |
| 1 | X₁₄ | Minimal lumen diameter | 0.5536 | 57.57 |
| 2 | X₁₄ * X₁₆ | Minimal lumen diameter | 0.2985 | 31.04 |
| | | & Proximal reference | | |
| | | lumen diameter | | |
| 3 | X₁₀ * X₁₅ | Calculated % RCA & | 0.0583 | 6.06 |
| | | Distal reference | | |
| | | lumen diameter | | |
| 4 | X₁₀ * X₁₆ | Calculated % RCA & | 0.0404 | 4.20 |
| | | Proximal reference | | |
| | | lumen diameter | | |
| 5 | X₁ * X₁₆ | Age & Proximal reference | 0.0048 | 0.50 |
| | | lumen diameter | | |
| 6 | X₁ | Age | 0.0022 | 0.23 |
| 7 | X₈ | Body surface area, m² | 0.0017 | 0.18 |
| 8 | X₁ * X₇ | Age & Body mass | 0.0011 | 0.11 |
| | | index, kg/m² | | |
| 9 | X₁₈ | Distance to Ostium (OS) | 0.0008 | 0.08 |
| | | from MLD | | |
| 10 | X₃ | Hypertension | 0.0002 | 0.02 |
| | | (High blood pressure) | | |
| Total | | | 0.9616 | 100 |

### Validating the Proposed Models: The Proposed Analytical Model

To validate the proposed model, as outlined in the equation, it is crucial to ensure that all underlying assumptions are met. The first assumption, linearity, is examined through the presentation of a linearity plot, specifically the partial residual plot, depicting the relationship between the response variable and the significant attributable risk factors.

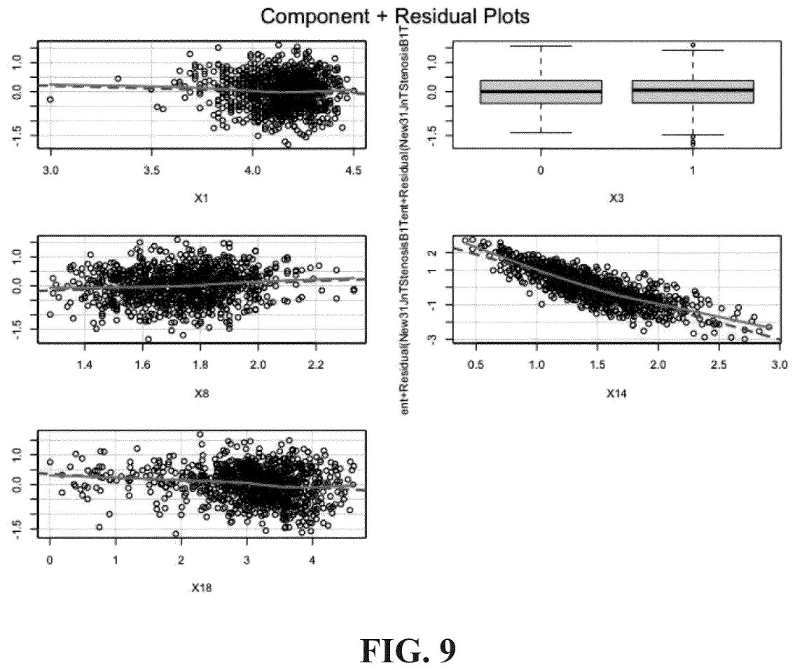

FIG. 9 illustrates a clear and well-established linear relationship, as evidenced by the coherent patterns of the blue and pink lines. Additionally, the inventors addressed the issue of an insignificant intercept term in the initial model, and this has been resolved in the transformed model proposed. The p-value of p-value=2e−¹⁶ (i.e. rejecting H₀: φ=0) confirms the linearity assumption of the model.

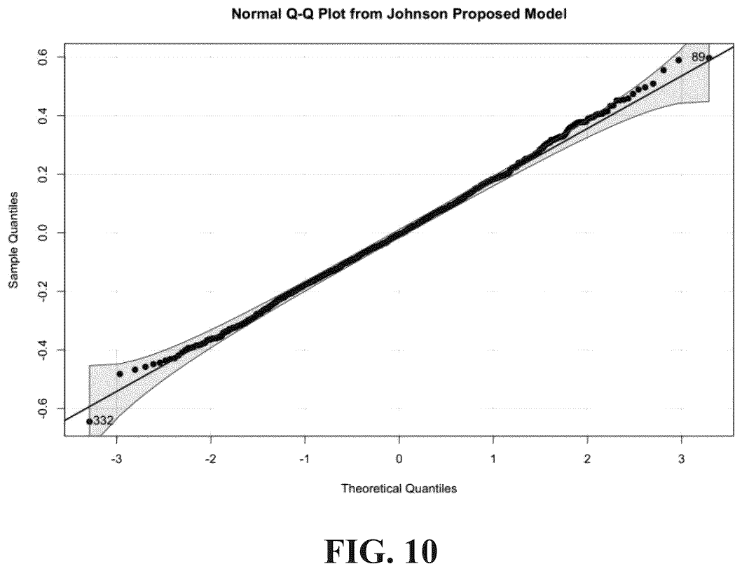

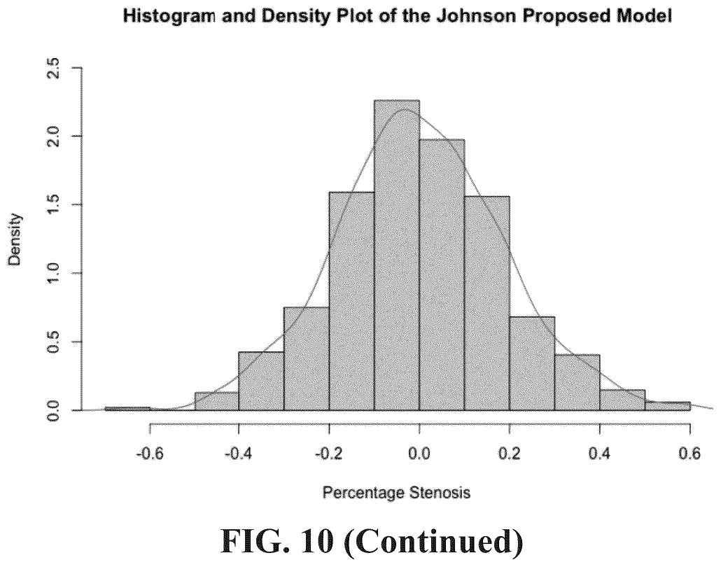

To assess the normality assumption of the proposed model, the inventors examined the distribution of residuals through normal plots, illustrated in FIG. 10. The first panel displays the normal Q-Q plot of residuals with 95% confidence bounds, while the second panel showcases the distribution of studentized residuals. Both panels demonstrate the adherence of the proposed model to the normality assumption, as all residual points fall within the 95% bound of the Q-Q plot with no major outliers.

To further substantiate these findings, the inventors conducted formal tests for normality: the Shapiro-Wilk's test yielded a substantial p-value of 0.5125, affirming that the residuals of the proposed model exhibit a normal distribution.

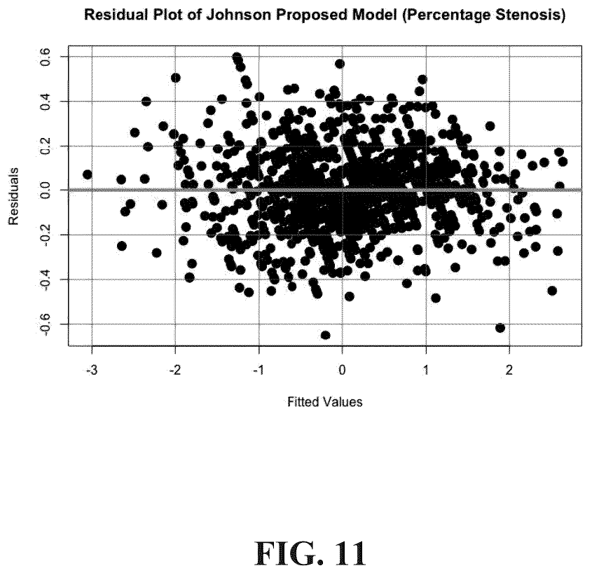

Homoscedasticity, an assumption asserting constant variance of residual errors, is a fundamental criterion met by our proposed model. To assess homoscedasticity, the inventors generated a plot of residuals against the fitted values, as depicted in FIG. 11. The residuals of the proposed model exhibit homoscedasticity, as there is no apparent pattern or trend, and no major outliers are observed, the points are randomly dispersed evenly above and below the zero lines without significant outliers. This observation is further supported by a non-constant variance score test, with a p-value of 0.157, surpassing the 0.05 significance level, affirming the fulfillment of the homoscedasticity assumption.

The inventors also conducted the Durbin-Watson test to examine the presence of autocorrelation among residuals. The test resulted in a p-value=0.08, signifying that the residuals are uncorrelated and that there is no indication of no serial correlation of the error term. This assumption is satisfied.

As highlighted earlier in the model-building process, multicollinearity posed a challenge to the model. While some argue that multicollinearity may not significantly impact the performance of model prediction, it is generally anticipated that a statistical model with minimal or no multicollinearity will outperform models with high multicollinearity. Multicollinearity has the potential to substantially increase the mean square error (MSE) and result in unstable coefficients for some attributable variables. These variables may become statistically insignificant, despite their importance in predicting the response. A common strategy to address multicollinearity involves eliminating redundant predictors highly correlated with other predictors.

The inventors then checked the Variance Inflation Factor (VIF) of the significantly identified risk factors in the model. VIF is a measure of the amount of multicollinearity present among variables. The VIF score of less than 10 implies that the data set has no multi-collinearity effect among the attributable variables. The inventors observed from Table [3] that the VIF value for each attributable risk factor is far less than 10 and thus, our data set passed the multicollinearity test.

TABLE 3
Variance Inflation Factor (VIF) of Significant Risk Factors

| | Risk Factor | | | | | | | | |
|---|---|---|---|---|---|---|---|---|---|
| | X₁ | X₃ | X₇ | X₈ | X₁₀ | X₁₄ | X₁₅ | X₁₆ | X₁₈ |
| VIF | 1.69 | 1.04 | 1.30 | 1.32 | 1.44 | 2.02 | 2.70 | 1.28 | 1.03 |

### Evaluation of the Proposed Models

To evaluate the performance of our proposed model, the inventors have to validate the prediction accuracy in terms of predicting percentage stenosis in the arteries of the heart in patients with coronary artery stenosis.

### The Proposed Analytical Model

The proposed analytical model's prediction accuracy is evaluated by examining the achieved R2 after its development. The R2 values indicate the proportion of variation in the response variable ( [symbol] ) explained by the relationship between the five (5) significant individual risk factors and the five (5) interaction terms. A higher R² signifies a better goodness-of-fit for the model. The analytical model proposed demonstrates high quality, boasting an R² of 96%, and successfully validates all key assumptions.

To further assess the model's quality, the root mean square error (RMSE) is employed to gauge the difference between predicted and observed values, as given by:

$$RMSE = \sqrt{\frac{1}{n} {\sum }_{i = 1}^{n} {( {}_{p} {S}_{i} \_ )}^{2}} .$$

A smaller RMSE indicates greater accuracy, representing the standard deviation around predicted values. In this instance, the proposed Johnson transform analytical model yields an RMSE of 0.188. This suggests a 95% confidence that our predicted values fall within +/−0.188≈0.2 of the actual values. Such high accuracy underscores the reliability of the model in predicting the percentage stenosis in the arteries of the heart for patients with coronary artery stenosis or new patients seeking information about their stenosis degree or severity.

Secondly, the Mean Absolute Error (MAE) stands out as another valuable metric for model evaluation. MAPE is calculated by:

$$MAE = ( \frac{1}{n} {\sum }_{i} \vert \frac{{}_{p} {S}_{i} -}{{}_{p} {S}_{i}} \vert )$$

The MAE, computed based on observed and predicted values, is 0.147. This small value affirms the high quality of the proposed model given the nature of the data.

Additionally, the inventors conducted a Kruskal-Wallis test to compare the observed and predicted returns, as outlined in Table [4]. Our null hypothesis (H₀) states that there is no significant statistical difference between the two percentage stenosis values. With a p-value of 0.9869 exceeding 0.05, the inventors failed to reject the null hypothesis, concluding with 95% confidence that there is no significant difference between the observed and predicted percentage stenosis.

TABLE 4
Kruskal-Wallis rank sum test of the Difference in Observed and
Predicted Percentage Stenosis in the Artery of the Heart

| | | Data: list(Observed, |
|---|---|---|
| Type of Test | Percentage Stenosis | Predicted) |
| Kruskal-Wallis | chi - squared(\tilde{χ}²) = | p - value = 0.9869 |
| | 2.716e⁻⁰⁴ | |

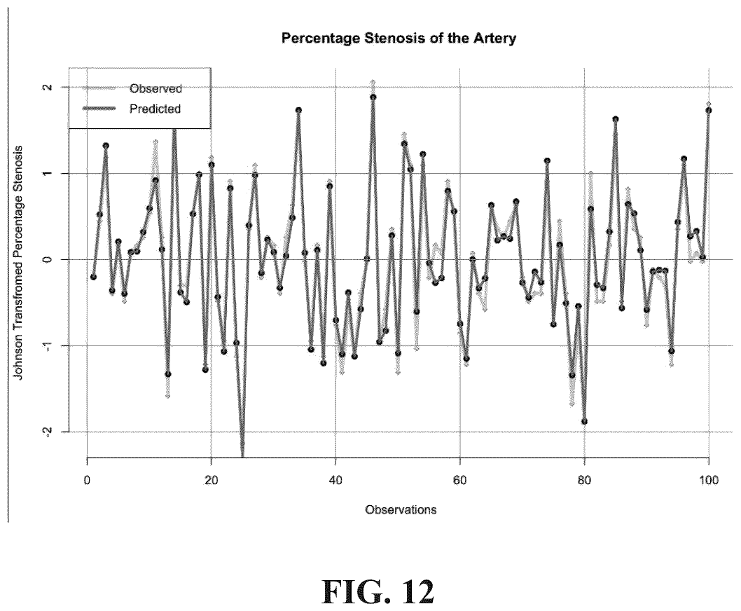

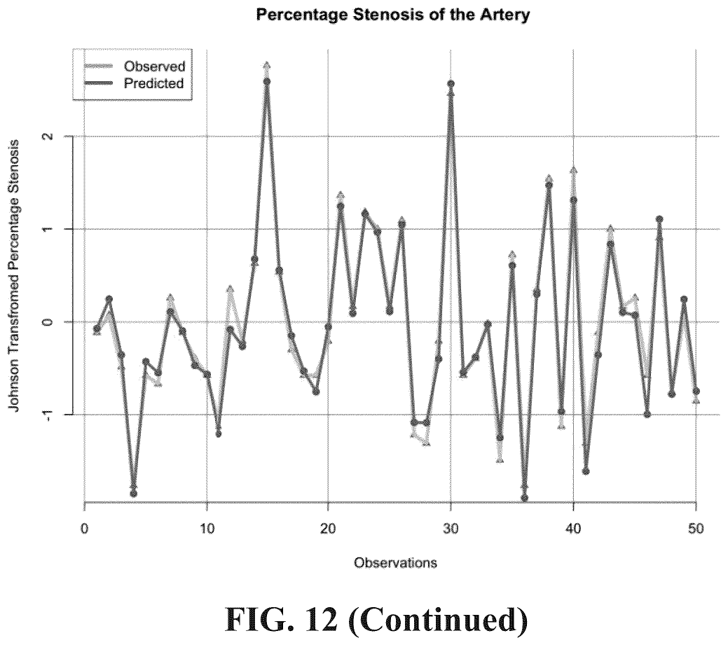

To validate the above evaluations of our proposed analytical models, the inventors trained 80% of the data to build our model and tested the prediction accuracy on the remaining 20% test data. FIG. 12 provides a graphical representation of this process, showcasing randomly plotted sets of 100 and 50 predicted values alongside their corresponding observed values. The pronounced alignment between the purple line representing predicted values and the green line representing observed values indicates a model with high prediction accuracy, efficiency, precision, and robustness. This observation attests to the capability of our model not only to discern inherent patterns and underlying trends within the data but also to predict percentage stenosis values with a high degree of accuracy. The close alignment between predicted and observed values consistently reinforces the reliability and effectiveness of the proposed model. It serves as compelling evidence for the model's accuracy in predicting the percentage stenosis in the arteries of the heart of patients with coronary artery stenosis and new patients inquiring about the severity of their stenosis.

The findings revealed a high level of correlation between the predicted and test set values for both the trained model and the entire data model, with correlation coefficients reaching 0.9806. A near-perfect correlation of 0.9806≈1 between the predictions of the two models underscores the robustness of the proposed approach.

TABLE 5
Prediction Comparison of Percentage Stenosis
Based on Train and Test Models

| | |
|---|---|
| Percentage | Predicted |
| Stenosis | Percentage Stenosis |

| Observation | Observed | Entire data | Trained |
|---|---|---|---|
| Number | Values | Model | Model |
| 964 | 0.999 | 1.013 | 1.022 |
| 475 | 0.630 | 0.571 | 0.581 |
| 308 | 1.718 | 1.687 | 1.694 |
| 66 | −0.851 | −0.653 | −0.659 |
| 794 | 0.999 | 1.157 | 1.179 |
| 1003 | −0.114 | −0.093 | −0.092 |
| 888 | 0.723 | 0.804 | 0.806 |
| 190 | 0.072 | 0.099 | 0.099 |
| 918 | 1.453 | 1.449 | 1.443 |
| 274 | 0.999 | 1.260 | 1.259 |
| 597 | 2.141 | 2.154 | 2.164 |
| 50 | −1.034 | −0.714 | −0.716 |
| 485 | 0.908 | 1.107 | 1.123 |
| 610 | 0.908 | 0.779 | 0.786 |
| 1129 | 0.164 | 0.111 | 0.113 |
| 471 | 0.537 | 0.542 | 0.553 |
| 263 | 0.907 | 0.877 | 0.890 |
| 402 | −1.125 | −1.123 | −1.144 |
| 855 | 1.718 | 1.732 | 1.730 |

Lastly, Table [5] presents 20 randomly selected sets of predicted percentage stenosis from the 20% test data, after the inventors developed the model using the entire data and then 80% train data. The small difference in the values obtained reaffirms the high quality, efficiency, predictive accuracy, and precision of the proposed Johnson analytical model. The proposed Johnson transform nonlinear analytical model boasts a high R₂ of 0.9616 (96.16%) and R_{adj}² of 0.9612 (96.12%). This implies that the model can predict the percentage stenosis in the arteries of the heart for patients with coronary artery stenosis or new patients seeking information about their stenosis degree or severity with a high degree of accuracy, at least 96%. The root mean square error (RMSE) of the proposed Johnson transform nonlinear analytical model (+/−0.188≈0.2). This suggests that, over a range of prediction values, the values of the proposed Johnson transform nonlinear analytical model will slightly deviate above or below the original values by approximately 0.2 95% of the time.

### Discussion: Usefulness of the Identified Significant Risk Factors

Minimal Lumen Diameter (MLD) (X₁₄), a measure of the narrowest point within a blood vessel like the artery, holds the top rank as the number one contributor that drive the PSAH. MLD provides valuable insights into the extent of narrowing or blockage in the vessel. Smaller MLD values are associated with an increased risk of future cardiovascular events, such as heart attacks or strokes.

The interaction between minimal lumen diameter and Proximal reference lumen diameter (X₁₄*X₁₆): A smaller MLD, along with a narrowest PRLD, can indicate a more severe stenosis, meaning a higher degree of narrowing and a greater risk of cardiovascular events. Conversely, a larger MLD, along with a wider PRLD, can indicate a less severe stenosis, meaning a lower degree of narrowing and a lower risk of cardiovascular events. MLD and PRLD can be used to help interpret FFR measurements where the pressure difference across a stenosis is compared to the pressure in a normal vessel.

The interaction between calculated % RCA and Distal reference lumen diameter (X₁₀*X₁₅): Calculated % RCA (relative coronary area) and Distal reference lumen diameter (DRLD) identified as an interaction risk factor in the model. Research suggests that both are used as indicators of stenosis severity. The higher the calculated % RCA and the narrower the DRLD, the more severe the stenosis. This can be controlled through medical treatments such as medication or stenting, which aim to reduce the narrowing of the artery and improve blood flow. Additionally, lifestyle modifications like exercise and a healthy diet can help improve blood flow and reduce the risk of further narrowing.

The interaction between calculated % RCA and Proximal reference lumen diameter (X₁₀*X₁₆): Proximal reference lumen diameter (PRLD) is another key measure of stenosis severity, and when combined with calculated % RCA, it can help to paint a more accurate picture of the overall severity of the stenosis. A narrowing of the proximal reference lumen diameter, along with a high calculated % RCA, indicates a more severe stenosis and may indicate a higher risk of cardiovascular events like heart attacks or strokes. Conversely, a wider proximal reference lumen diameter, along with a lower calculated % RCA, may indicate a less severe stenosis with a lower risk of cardiovascular events. It's important to note that these two measurements can provide valuable information when used in conjunction with other diagnostic tools and factors.

The interaction between Age (X₁) and Proximal reference lumen diameter (X₁*X₁₆): As people age, their arteries can become stiffer and narrower, leading to a decrease in PRLD. This process, called arterial stiffening or arteriosclerosis, is a natural part of aging and is influenced by a variety of factors. Arterial stiffening reduces the ability of the arteries to expand and contract with the flow of blood, which can decrease PRLD and increase the risk of further narrowing the artery and exacerbating stenosis.

Age (X₁) can be seen interacting with two distinct risk factors and standing as a significant risk factor itself. Age is recognized as a major risk factor for atherosclerosis, the underlying process leading to stenosis. As individuals age, their arteries become less flexible and more susceptible to plaque buildup. The prevalence of atherosclerosis, and consequently stenosis, steadily increases with age.

Body Surface Area (X₈) is a measure that takes into account a person's height and weight to estimate the total skin area. It is often used in clinical settings to calculate drug dosages and to assess cardiac output, thus providing a way to adjust medication dosages and interpret test results in patients, as it accounts for differences in body size and shape. The relationship between body surface area (BSA) and stenosis is a bit more complicated than BMI. Research on the relationship between BSA and stenosis suggests that people with larger body sizes tend to have larger blood vessels, which can put them at greater risk for atherosclerosis (the buildup of plaque in the arteries). So while BSA itself is not a direct risk factor for stenosis, factors that contribute to changes in BSA, such as weight gain or loss, could indirectly influence the percentage stenosis in the arteries of the heart.

The interaction between age and body mass index, kg m² (X₁*X₇): As people age, their body composition changes. This combination of aging and obesity increases the risk of developing stenosis, as the stiffening of the arteries and the accumulation of fat can further narrow the blood vessels. Here is how different age and weight combinations can affect the risk of percentage stenosis: Older people who are obese are at higher risk of increasing percentage stenosis than younger people who are obese. This is because aging and obesity act together to increase inflammation, which can accelerate the development of atherosclerosis and lead to the narrowing of blood vessels. Younger people who are obese may still be at higher risk of increasing percentage stenosis than older people who are not obese. This is because obesity is a major risk factor for developing cardiovascular disease, even at a younger age. However, a young person who is not obese may still be at some risk of developing stenosis due to other risk factors such as high blood pressure or high cholesterol.

The distance between the Minimum Lumen Diameter (MLD) and the Ostium (OS) (Xis), also known as the “os-MLD gap”, is one factor that can be used to assess the risk of increasing percentage stenosis. A smaller gap between the MLD and OS indicates a greater risk of percentage stenosis. This is because when the gap is smaller, the blood flow through the artery is more constricted, which can lead to an increase in pressure and a decrease in blood flow. Additionally, a smaller gap can also indicate that the artery is more narrow overall, which can also contribute to the development of percentage stenosis. If the distance between MLD and OS decreases, it can cause turbulence in the blood flow, which can increase the risk of blood clots and other complications that can lead to percentage stenosis.

BMI (X₇) is a measure of body fat based on an individual's weight and height. High BMI is often associated with an increased risk of cardiovascular diseases such as heart attack or stroke. Excessive body weight can contribute to conditions such as hypertension, high cholesterol, and diabetes, which are risk factors for heart diseases, including coronary artery disease. A higher BMI is linked to the development of atherosclerosis, a condition characterized by the buildup of plaque in the arteries. This plaque formation can lead to stenosis, narrowing the arteries, and reducing blood flow to the heart. This is because obesity can increase inflammation and plaque buildup in the arteries. For every 1-point increase in BMI, the risk of coronary artery disease increases by about 4%. This means that individuals with a BMI of 30 or higher (considered obese) have a much higher risk of coronary artery disease than those with a BMI in the normal range (18.5 to 24.9). Lifestyle factors associated with a high BMI, such as poor dietary habits and a lack of physical activity, contribute to the progression of atherosclerosis.

Hypertension (X₃), ranking last (10th) among the risk factors, represents the medical term for high blood pressure. This condition occurs when the force of blood against the walls of blood vessels is higher than normal. Hypertension is a critical risk factor for cardiovascular disease, capable of causing damage to the heart and blood vessels over time. Various factors, including diet, lifestyle, certain medications, and chronic conditions, can contribute to high blood pressure.

### Useful Contributions

The inventors have identified the following five (5) risk factors that individually significantly contribute to the percentage stenosis of the arteries of the heart, PSAH: 1) Minimal lumen diameter, 2) Age, 3) Body surface area, 4) Distance to Ostium (OS) from MLD, 5) Hypertension (High blood pressure).

The inventors have identified five (5) interactions of the risk factors that significantly contribute to the percentage stenosis of the arteries of the heart, PSAH: 1) Age & Body mass index, 2) Minimal lumen diameter & Proximal reference lumen diameter, 3) Calculated % RCA & Distal reference lumen diameter, 4) Age & Proximal reference lumen diameter, 5) Calculated % RCA & Proximal reference lumen diameter

The inventors have identified the percentage of contribution that each of the individual risk factors and interactions contribute to the percentage stenosis of the arteries of the heart, PSAH (identified in Table 6).

TABLE 6
Rank of the Significant Attributable Risk Factors
and Interaction Terms based on their percentage
of contribution to the percentage stenosis

| Rank | Variable | R² | % Contribution |
|---|---|---|---|
| 1 | Minimal lumen diameter | 0.5536 | 57.57 |
| 2 | Minimal lumen diameter & | 0.2985 | 31.04 |
| | Proximal reference lumen diameter | | |
| 3 | Calculated % RCA & Distal | 0.0583 | 6.06 |
| | reference lumen diameter | | |
| 4 | Calculated % RCA & Proximal | 0.0404 | 4.20 |
| | reference lumen diameter | | |
| 5 | Age & Proximal reference | 0.0048 | 0.50 |
| | lumen diameter | | |
| 6 | Age | 0.0022 | 0.23 |
| 7 | Body surface area, m² | 0.0017 | 0.18 |
| 8 | Age & Body mass index, kg/m² | 0.0011 | 0.11 |
| 9 | Distance to Ostium (OS) from MLD | 0.0008 | 0.08 |
| 10 | Hypertension (High blood pressure) | 0.0002 | 0.02 |
| Total | | 0.9616 | 100 |

Given the information on the risk factors identified, the proposed real data-driven analytical model will predict the percentage stenosis of the arteries with a high degree of accuracy. Finally, all of the above very important findings are given with at least 96% accuracy.

### Example: Phase H—Optimization Method for the Percentage Stenosis of the Arteries of the Heart, (PSAH)

### Overview

In Phase I of our innovation, the inventors have developed a highly accurate, real data-driven predictive model that identifies the risk factors that drive the percentage of stenosis in the arteries of the heart. In Phase II of our innovation, the inventors utilized the analytical predictive model to identify the actual values of the risk factors (target values) that will minimize the percentage stenosis of the arteries of the heart to help reduce experiencing a heart attack or stroke. In addition to developing the step-by-step method to optimize the percentage stenosis of the developed predictive model, the inventors have also developed a method of obtaining confidence limits for the true percentage stenosis of the artery of a given arterial stenosis patient. In summary, our innovation in Phase II constitutes the following important findings: For the 10 given risks, factors, and interactions that the inventors have established in Phase I of our innovation, the inventors developed a method of identifying the values of the risk factors (target values) that would optimize (minimize) the percentage stenosis of the arteries of the heart for a given arterial stenosis patient. The inventors have developed a method of obtaining with at least 95% confidence that the true percentage stenosis of the arteries is within two values: an upper bound and a lower bound. The inventors have proceeded to apply the method that the inventors developed for patients of one grading stage of severity to two additional classifications, namely mild and moderate stenosis, of the percentage stenosis of an arterial stenosis patient. The inventors have developed a process of 2-D graphical visualization that characterizes the behavior of the percentage stenosis of the arteries of the heart as a function of the risk factors that the inventors have identified to cause the problem. The above important and useful findings are presented with a high degree of accuracy.

### Introduction

In Phase I of our innovation, the inventors developed an analytical predictive model for the percentage of arterial stenosis in patients with coronary artery stenosis. The inventors conducted a thorough analysis of the risk factors that influence the percentage stenosis using real data consisting of 1,132 stable and unstable angina patients with eighteen (18) risk factors. Our model identified 10 risk factors, whose combination produced significant individual attributable risk factors and interaction terms that predict the percentage of arterial stenosis in patients with at least 96% accuracy. The inventors ranked these risk factors based on their contribution to the percentage stenosis, and this information raises a very important question: how can the inventors optimize (minimize) the percentage stenosis using the identified risk factors?Specifically, what are the target or optimal values of each identified significant risk factor that will help us achieve minimal percentage stenosis values in the arteries of the heart of patients?

The inventors develop a robust and efficient method for optimizing (minimizing) the percentage stenosis of the arteries of the heart. The inventors use our statistical predictive model and performed an optimization analysis using the desirability function approach of response surface methodology (RSM). RSM combines the design of experiments, regression models, and optimization to evaluate the relationship between the risk factors and the response variable. This allows for optimal outcomes to be achieved by identifying the most influential risk factors and their optimal levels. By utilizing this methodology, the inventors the were able to minimize the percentage stenosis in the arteries of the heart of an arterial stenosis patient while ensuring a reliable and efficient method.

The primary objective of RSM is to optimize response variables by maximizing, minimizing, or obtaining a target value. It achieves this by identifying the best values of controllable risk factors that lead to optimal responses. Optimization in RSM can be classified into three categories: Single Response Surface Optimization (SRSO), Dual Response Surface Optimization (DRSO), and Multiple Response Surface Optimization (MRSO). SRSO optimizes the mean response based on a combination of independent variables subject to constraints. DRSO, on the other hand, optimizes one response subject to the constraints of the other response, taking into account the trade-offs between the two competing responses. MRSO optimizes one mean response based on the trade-offs among one or more responses while adhering to the constraints.

In RSM, the inventors utilized two major approaches for response optimization: the Constraint Optimization Problem Approach (COPA) and the Desirability Function Approach (DFA). The COPA seeks to optimize responses by formulating the problem as a constrained optimization problem. In contrast, DFA optimizes the responses by using a desirability function that characterizes the desired response and its target value. This method allows for the consideration of multiple responses and the identification of optimal combinations of risk factors that lead to the desired responses.

The desirability function is a method used for risk factor optimization. The Desirability Function Approach (DFA) is a response optimization strategy that optimizes the response as a function of controllable input risk factors. The combined use of RSM and the DFA approach results in a powerful method for finding optimal dynamics between responses. This combination is known as the “Desirability Optimization Methodology” and is utilized in our innovation to achieve our objective.

In developing our method, the inventors used a Single Response Surface Optimization (SRSO) type and utilized the Desirability Function Approach (DFA) to minimize the percentage stenosis in the arteries of the heart of patients with coronary artery stenosis. To validate the efficiency of our optimization method, the inventors use various statistical processes, including the value of the desirability function, R², R²_{adj}, and R²_{pred} statistic, as the well as obtaining the 95% confidence interval (CI) and prediction interval (PI) of the optimal point of the percentage stenosis. By using these measures, the inventors can ensure the accuracy and reliability of our results and confirm the effectiveness of our approach in minimizing the percentage stenosis in the arteries of the heart of patients with coronary artery stenosis.

The heart lies peacefully in the chamber of the chest, with the size of a fist, the weighs about 300 grams, and beats 60-80 times per minute throughout our entire life. It pumps 5-6 liters of blood throughout our body. The blood carries oxygen and nutrients vital to the tissues, and it also carries waste products away from the tissues. If there is disruption of any of these processes for any reason, the body's organs begin to fail very quickly. So life itself is dependent on efficient, continuous operation. As atherosclerotic plaque builds up in the arteries of a person with heart disease, the inside of the arteries begins to narrow, which lessens or blocks the flow of blood. Plaque can also rupture (break open), and when this happens, a blood clot can form on the plaque, blocking the flow of blood and leading to a mismatch between myocardial oxygen supply and myocardial oxygen demand. This results in chest pain, shortness of breath, and fatigue, which are the most likely symptoms.

Percentage stenosis is a measure of the degree of narrowing of a blood vessel, usually an artery, expressed as a percentage of the vessel's original diameter. If a vessel's diameter is narrowed to 50% of its original size, then the percent diameter of stenosis is 50%,[1]. The most common disease, coronary artery heart disease, is caused by arterial stenosis.

### Data Description

The data employed for this phase precisely corresponds to the data used in Phase I of our innovation.

### Optimization Method of Percentage Stenosis of the Arteries of the Heart

### Overview of the Developed Method

The desirability function is a method in industry for optimizing multiple response processes.

The DFA is based on transforming all obtained responses to a scale-free value, called desirability, with values ranging from 0 to 1. A desirability value of 0 is assigned when the factors give an undesirable response, while a value of 1 corresponds to the optimal performance for the important factors. For each response, yᵢ(x), a desirability function dᵢ assigns values between 0 and 1 to the possible values of Yᵢ. A completely undesirable value of yᵢ is assigned a value of 0, while a completely desirable or ideal response value is assigned a value of 1. The individual desirabilities are then combined using the geometric mean to calculate the overall desirability, D. The DFA is used to optimize the response of a process by adjusting the controllable input factors to achieve the highest possible overall desirability value.

A response surface optimization problem with single or multiple responses, Y=, ŷⱼ and input variables or risk factors, X=x, can be expressed as follows:

Optimize [ŷ₁(x),ŷ₂(x), . . . , ŷₙ(x)], subject to z∈Ω
where ŷⱼ, for j=1, 2, . . . , n, is the estimated j^{th} response variable and n is the number of responses. For, j=1, means a single response problem, j=2, implies a dual response problem, and j=2 or more implies a multiple response problem. Ω is the experimental space of x.

The DFA method is used to identify the combination of controllable input risk factors that optimize the defined goal of the response variable(s). Different objectives of the response require the use of different desirability functions.

Optimization objectives can include maximizing, minimizing, or achieving a target value of the response variable. If the objective is to maximize the response variable (i.e., the larger the better), the desirability function can be defined as:

$${d}_{j} ( {\hat{y}}_{j} ) = \{ \begin{matrix}0 , & {\hat{y}}_{j} < {L}_{j} \\ {( \frac{{\hat{y}}_{j} - {L}_{j}}{{T}_{j} - {L}_{j}} )}^{α} , & {L}_{j} \leq {\hat{y}}_{j} \leq {T}_{j} \\ 1 , & {\hat{y}}_{j} > {T}_{j}\end{matrix} \qquad ( 9 )$$

If the objective is to minimize the response (i.e. the smaller the better), the desirability function is defined as

$${d}_{j} ( {\hat{y}}_{j} ) = \{ \begin{matrix}1 , & {\hat{y}}_{j} < {T}_{j} \\ {( \frac{{U}_{j} - {\hat{y}}_{j}}{{U}_{j} - {T}_{j}} )}^{α} , & {T}_{j} \leq {\hat{y}}_{j} \leq {U}_{j} \\ 0 , & {\hat{y}}_{j} > {U}_{j}\end{matrix} \qquad ( 10 )$$

And, if the objective is to obtain a target value of the response, the two-sided desirability function is defined as

$${d}_{j} ( {\hat{y}}_{j} ) = \{ \begin{matrix}0 , & {\hat{y}}_{j} < {L}_{j} \\ {( \frac{{\hat{y}}_{j} - {L}_{j}}{{T}_{j} - {L}_{j}} )}^{{α}_{1}} , & {L}_{j} \leq {\hat{y}}_{j} \leq {T}_{j} \\ {( \frac{{U}_{j} - {\hat{y}}_{j}}{{U}_{j} - {T}_{j}} )}^{{α}_{2}} , & {T}_{j} \leq {\hat{y}}_{j} \leq {U}_{j} \\ 0 , & {\hat{y}}_{j} > {U}_{j}\end{matrix} \qquad ( 11 )$$

where α₁, α₂, and α represent the weighted parameters that define the shape of the individual desirability function, dⱼ(ŷⱼ). For α₁=α₂=1 implies the shape of dⱼ(ŷⱼ) is linearly increasing, α₁<1 and α₂<1 implies the shape of dj(\hat{ }yj) is concave, and α1>1 and α2>1 implies the shape of dⱼ(ŷⱼ) is convex. Tⱼ is the target value of ŷⱼ. Lⱼ and Uⱼ denote the lower bound and the upper bound of the response ŷⱼ, respectively. To obtain the overall desirability function, the inventors use the following equation:

$$D = {[ {\prod }_{j = 1}^{n} {d}_{j} {\hat{y}}_{j} ]}^{1 / n} = {[ {d}_{1} ( {\hat{y}}_{1} ) {d}_{2} ( {\hat{y}}_{2} ) … {d}_{n} ( {\hat{y}}_{n} ) ]}^{1 / n} . \qquad ( 12 )$$

D ranges from zero to one (i.e., 0≤D≤1). D=1 is the ideal case of optimality and indicates the optimum point of the response. Thus, the input variables, or risk factors, are very effective in optimizing the response. D=0 implies that the response variable(s) is/are outside the acceptable region, which means the controllable input factors are doing poorly in optimizing the response (the case of an undesirable response).

### The Method for Percentage Stenosis Optimization

In this innovation, the optimization problem involves a single response, which is to minimize the percentage of arterial stenosis in the heart. To optimize the response variable, the inventors utilized the desirability function defined by Equation [10]. Our optimization method followed the following steps: 1) Developing an analytical model that can significantly predict the continuous response/target variable(s), and risk factors with a high degree of accuracy, 2) Obtain the constraints or limits of the response(s) and input/attributable risk factors. 3) Identifying an appropriate desirability function that can optimize the response based on the response optimization objective, 4) Executing the function to obtain the optimal value(s) of the response(s), value(s) of the input risk factors, and value of the desirability function, dᵢ(ŷⱼ). 5) Validating the optimization method using the coefficient of variation R² and the adjusted R².

The results of the outlined optimization method for the percentage stenosis of arteries of the heart, PSAH, are as follows:

### Step 1

The developed analytical model, as presented in Phase I, that accurately predicts the percentage of arterial stenosis in the heart with at least 96% accuracy is given by:

$${}_{p} {S}_{T} = 2.1184 + 2.927 ln ( \frac{{}_{p} S - 4.3531}{4.8113 - {}_{p} S} ) . \qquad ( 13 )$$

$$and$$

$$= 4.3531 + \frac{0.4582}{1 + exp ( \frac{2.1184 - {}_{p} {S}_{T}}{2.937} )} \qquad ( 14 )$$

where γ=2.1184, η=2.9270, ξ=4.3531 and λ=0.4582

(−∞<γ<∞,γ−Johnson shape parameter,

η>0,η−Johnson shape parameter,

λ>0,ηλJohnson scale parameter,

−ξ<γ<∞, ξ−Johnson location parameter,
the equation of the final proposed model is given below:

$${}_{p} {𝒮}_{T} = 3.8921 - 0.4299 {X}_{1}^{′} - 2.1658 {e}^{- 02} {X}_{3} + 0.1102 {X}_{8} - 3.9886 {X}_{14} - 4.9892 {e}^{- 02} {X}_{18}^{′} - 2.1255 {e}^{- 03} {X}_{1}^{′} * {X}_{7} + 0.1649 {X}_{1}^{′} * {X}_{16} + 0.6529 {X}_{10} * {X}_{15} - 0.6194 {X}_{16} * {X}_{19} + 0.3572 {X}_{14} * {X}_{16} . \qquad ( 15 )$$

It should be noted that certain risk factors, while not significant contributors individually, were found to be significant when interacting with other risk factors. As such, these risk factors were included in the model for response optimization since they could not be separated.

In total, there were ten (10) significant attributable risk factors, including those that comprised the interaction terms.

### Step 2

The constraints, or lower and upper limits, of the percentage stenosis and the identified statistically significant risk factors are given in Table [10] below.

TABLE 7
The Constraints of the Response and Attributable Risk Factors.

| Percentage | Input or |
|---|---|
| Stenosis (%) | Risk Factors |
| 21 ≤ ₚS_{T} ≤ 86 | 20 ≤ X₁ ≤ 90 |
| | 0 ≤ X₃ ≤ 1 |
| | 13.2 ≤ X₇ ≤ 42.4 |
| | 1.28 ≤ X₈ ≤ 2.33 |
| | 0.35 ≤ X₁₀ ≤ 0.99 |
| | 0.41 ≤ X₁₄ ≤ 2.91 |
| | 1.53 ≤ X₁₅ ≤ 4.7 |
| | 1.77 ≤ X₁₆ ≤ 4.93 |
| | 1 ≤ X₁₈ ≤ 101.39 |

### Step 3

The optimization objective is to minimize the percentage stenosis in the arteries of the heart. Thus, the smaller the percentage stenosis, the better the degree of severity. Therefore, the inventors utilized the desirable function defined in equation [10].

### Step 4

After completing the optimization process, Table [8] below presents the optimal estimated response value (Minimal Stenosis), along with the values of the corresponding input/attributable risk factors and the desirability function value.

TABLE 8
Estimated Minimized Response with Optimal Values of Attributable
Risk Factors and the Desirability Function for Minimal Stenosis

| [symbol] | X₁ | X₃ | X₇ | X₈ | X₁₀ | X₁₄ | X₁₅ | X₁₆ | X₁₈ | [symbol] |
|---|---|---|---|---|---|---|---|---|---|---|
| 21 | 20 | 0 | 13.2 | 1.28 | 0.35 | 2.91 | 4.7 | 4.93 | 101.39 | 0.99 |

[symbol]

indicates data missing or illegible when filed

That is, if the risk factors achieve these target values, the inventors are assured that the patient will experience minimum stenosis.

### Step 5

Finally, the inventors have evaluated and validated the effectiveness of the optimization method in achieving the lowest target set for percentage stenosis using the desirability function value d(\hat{ }y), the coefficient of determination R² of the optimal model (which indicates the amount of variation in the response explained by the input factors), and the model prediction accuracy R²_{pred}, as shown in Table [9].

To determine the significance of the optimal response value, the inventors utilized a hypothesis test structured around the 95% confidence interval (CI) and prediction interval (PI).
H₀: the optimal value is significant.
vs.
Hₐ: the optimal value is not significant.

TABLE 9
Optimization Process Validation

| | |
|---|---|
| Estimated Minimum Stenosis | 21% |
| Desirability | 0.99 |
| R² | 96.16% |
| Adj. R² | 96.05% |
| R²(pred) | 95.89% |
| 95% CI | (11.5, 31.8) |
| 95% PI | (8.90, 33.1) |

That is, the inventors are at least 95% certain that the actual percentage stenosis of the arteries of the heart, PSAH, is between 11.5 and 31.8. Similarly, the predictive interval is between 8.90 and 33.1.

Additionally, the inventors produce estimates of target values of the percentage stenosis for patients who want to move from one grading stage of severity to another, along with the optimal value of the attributable risk factors, the results are given below:

### Mild Stenosis

TABLE 10
Estimated Minimized Response with Optimal Values of Attributable
Risk Factors and the Desirability Function for Mild Stenosis

| [symbol] | X₁ | X₃ | X₇ | X₈ | X₁₀ | X₁₄ | X₁₅ | X₁₆ | X₁₈ | [symbol] |
|---|---|---|---|---|---|---|---|---|---|---|
| 40 | 55 | 0 | 27.8 | 1.58 | 0.67 | 1.66 | 3.12 | 2.59 | 98.65 | 0.99 |

[symbol]

indicates data missing or illegible when filed

TABLE 11
Optimization Process Validation

| | |
|---|---|
| Estimated Minimum Stenosis | 40% |
| 95% CI | (26.54, 53.46) |
| Desirability | 0.99 |

That is, the inventors are at least 95% certain that the actual percentage stenosis of the arteries of the heart, PSAH, will be between 26.54% to 53.46%.

### Moderate Stenosis

TABLE 12
Esimated Minimized Response with Optimal Values of Attributable
Risk Factors and the Desirability Function for Moderate Stenosis

| [symbol] | X₁ | X₃ | X₇ | X₈ | X₁₀ | X₁₄ | X₁₅ | X₁₆ | X₁₈ | [symbol] |
|---|---|---|---|---|---|---|---|---|---|---|
| 60 | 66 | 0 | 13.6 | 1.29 | 0.98 | 0.41 | 1.62 | 1.77 | 84.47 | 0.99 |

[symbol]

indicates data missing or illegible when filed

TABLE 13
Optimization Process Validation

| | |
|---|---|
| Estiniated Minimum Stenosis | 60% |
| 95% CI | (44.91, 76.09) |
| Desirability | 0.99 |

Similarly, the inventors are at least 95% certain that the actual percentage stenosis of the arteries of the heart, PSAH, will be between 44.91% and 76.09%.

### Graphical Visualization of the Optimization Method

Graphical Visualization of the optimization method comes with 2-D contour plots of the combination of two risk factors in optimizing (minimizing) the response while holding other factors constant. By plotting the response against one or more attributable risk factors, these plots can help us understand how changes in these risk factors impact the percentage stenosis of the arteries of the heart, PSAH. For example, contour plots show the response surface as a two-dimensional plane, with points of similar response connected by contour lines of constant response.

In our proposed analytical predictive model, the inventors have identified five (5) significant individual risk factors and five (5) interaction terms that can impact the percentage stenosis of the arteries of the heart, PSAH. While it may be difficult to control all of these input variables in practice, understanding their relationship with the percentage stenosis of the arteries of the heart, PSAH can help individuals and health professionals minimize the percentage stenosis in the arteries of the heart. By analyzing the surface plots, Health professionals can identify the most effective combinations of input risk factors to minimize the percentage stenosis of the arteries of the heart, PSAH to the desired level. Thereby reducing the likelihood or expectation of experiencing a cardiovascular event (such as a heart attack or stroke) at or below a specified percentage of arterial stenosis in patients with coronary artery stenosis.

In addition, response surface plots can also provide guidance on how to achieve a target value of the PSAH while controlling for risk factors. By identifying the relationship between input risk factors and the PSAH, individuals and health professionals can make constructive decisions on how to manage and mitigate the risk in order to achieve their desired level of percentage stenosis. Overall, the use of PSAH surface plots can provide valuable insights into the behavior of input variables and their impact on the PSAH. By leveraging this knowledge, patients can optimize their percentage stenosis and minimize their value.

The inventors utilized 2-D contour plots to explore the optimal combination of two input/risk factors that minimize the percentage stenosis while holding other factors constant. The contour plot displays the deep green color region, which corresponds to the higher percentage stenosis, with deeper green colors indicating proximity to the less optimal point and light green indicating proximity to the desirable optimal point.

In contrast, the light blue to deep blue regions indicate a decreasing percentage stenosis, which is the desirable optimal point. Please note that not all combinations of attributable risk factors are presented in these plots. Our focus is on the major risk factors that contribute significantly to the percentage stenosis, as defined in Phase I of our innovation.

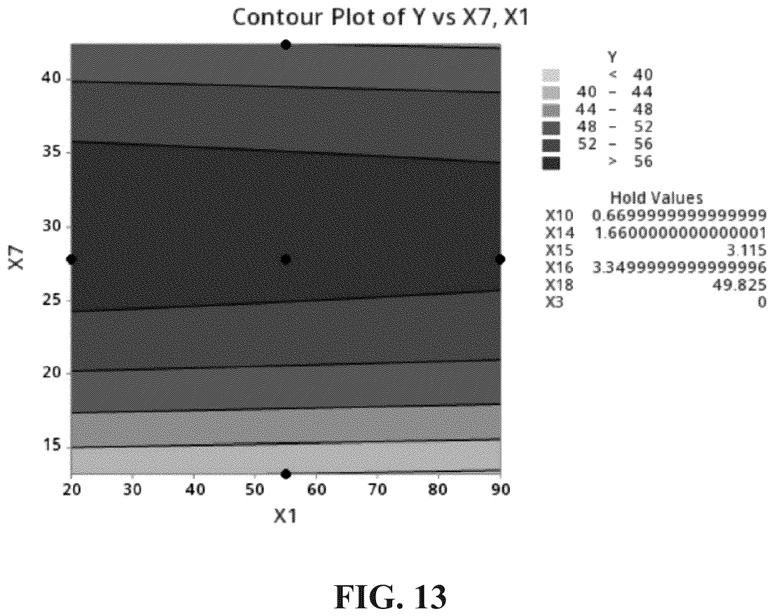

FIG. 13 displays the contour plot of Age (X₁) and Body Mass Index, kg m² (X₇) which reveals that the optimal (minimal) percentage stenosis in the arteries of the patient, PSAH can be achieved at almost all levels of age and a low-level body mass index below 15 kg/m². As the level of body mass index increases, the percentage stenosis increases at all levels of age groups. Higher levels of percentage of stenosis are obtained at all levels of age group when the body mass index goes beyond 25 kg/m².

This finding aligns with the literature that the combination of aging and obesity increases the risk of developing stenosis, as the stiffening of the arteries and the accumulation of fat can further narrow the blood vessels. This is because aging and obesity act together to increase inflammation, which can accelerate the development of atherosclerosis and lead to the narrowing of blood vessels. Younger people who are obese are still at higher risk of increasing percentage stenosis as well as all the older people who are obese.

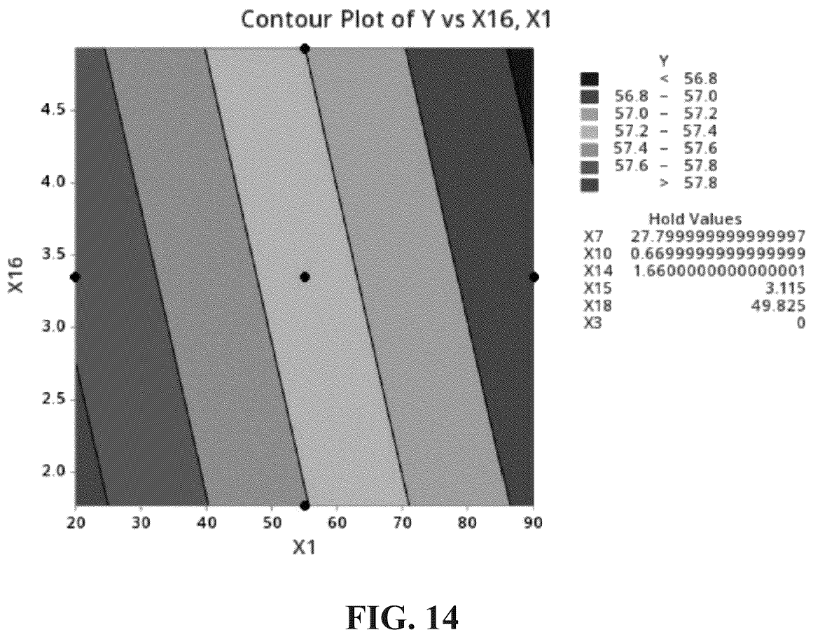

FIG. 14 displays the contour plot of Age (X₁) and proximal reference lumen diameter (X₁₆) which reveals that the optimal (minimal) percentage stenosis in the arteries of the patient can be achieved at a higher age level and a larger proximal reference lumen diameter. The inventors observe the highest percentage of stenosis in the younger age group, less than 40, and a smaller level of proximal reference lumen diameter below 3.0 mm.

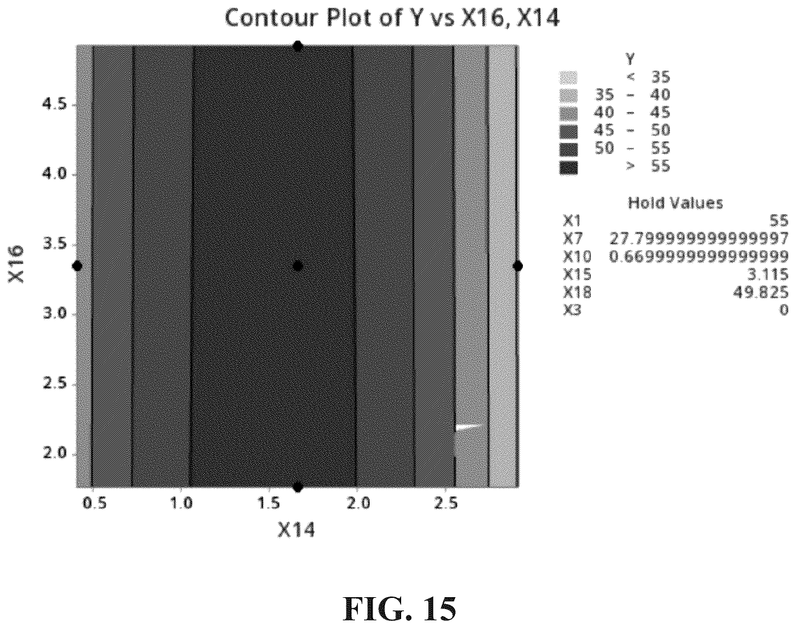

FIG. 15 displays the contour plot of the Minimal Lumen Diameter(X₁₄) and proximal reference lumen diameter (X₁₆) which reveals that the optimal (minimal) percentage stenosis in the arteries of the patient can be achieved at a higher value of the minimal lumen diameter (X₁₄) beyond 2.5 mm and at all values of the proximal reference lumen diameter. The inventors observe the highest percentage of stenosis at 1.0 mm to 2.0 mm of minimal lumen diameter and at all values of the proximal reference lumen diameter.

This finding is consistent with the medical argument since MLD and PRLD are used to help interpret FFR measurements where the pressure difference across a stenosis is compared to the pressure in a normal vessel, a smaller MLD, along with a narrower PRLD, can indicate a more severe stenosis, meaning a higher degree of narrowing and a greater risk of cardiovascular events. Conversely, a larger MHLD, along with a wider PRLD, can indicate a less severe stenosis, meaning a lower degree of narrowing and a lower risk of cardiovascular events.

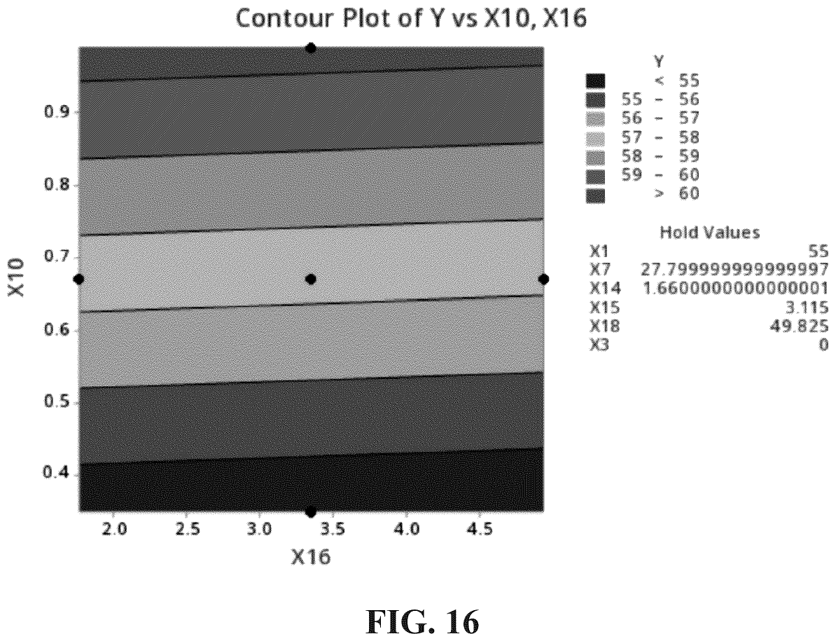

FIG. 16 displays the contour plot of the Calculated % RCA(X₁₀) and proximal reference lumen diameter (X₁₆) which reveals that the optimal (minimal) percentage stenosis in the arteries of the patient can be achieved at low levels of calculated % RCA below 0.5% and consistent levels of proximal reference lumen diameter. The highest degree of percentage stenosis is obtained at higher levels of calculated % RCA beyond 0.9% and consistent levels of proximal reference lumen diameter.

This finding is consistent with medical argument: a high calculated % RCA indicates a more severe stenosis and may indicate a higher risk of cardiovascular events like heart attacks or strokes. Conversely, a wider proximal reference lumen diameter, along with a lower calculated % RCA, may indicate a less severe stenosis with a lower risk of cardiovascular events.

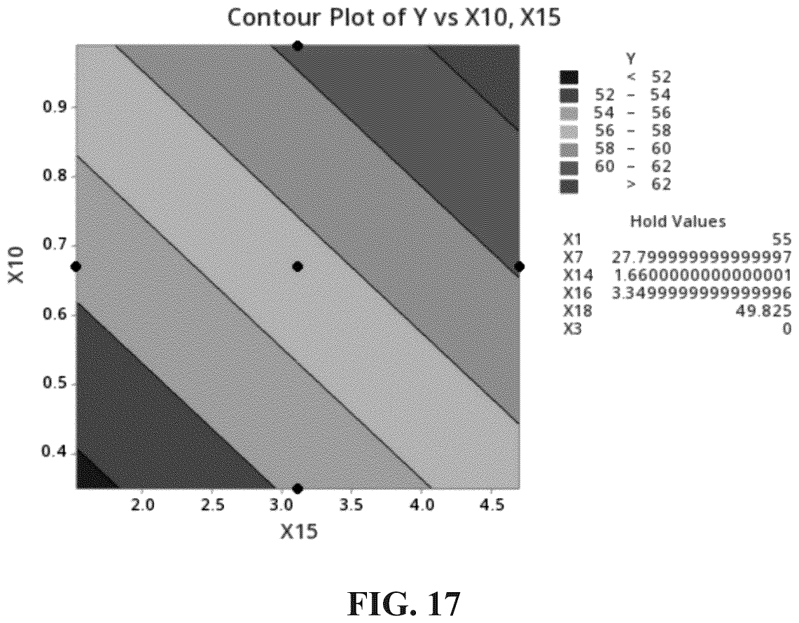

FIG. 17 displays the contour plot of the Calculated % RCA(X₁₀) and distal reference lumen diameter (Xis) which reveals that the optimal (minimal) percentage stenosis in the arteries of the patient can be achieved at both low levels of calculated % RCA below 0.4% and low levels of proximal reference lumen diameter below 2.0 mm. The highest degree of percentage stenosis is obtained at both higher levels of calculated % RCA beyond 0.9% and higher levels of proximal reference lumen diameter beyond 4.5 mm.

This finding aligns with the medical argument that the interaction between calculated % RCA and distal reference lumen diameter identified as an interaction risk factor in the model increases the percentage stenosis. Research suggests that both are used as indicators of stenosis severity. The lower the calculated % RCA and the narrower the DRLD, the more severe the stenosis.

It's important to note that these measurements in our innovation can provide valuable information when used in conjunction with other diagnostic tools and factors. They help to paint a more accurate picture of the overall severity of the stenosis. To achieve the desired target values of these risk factors and obtain optimal values for our response, the inventors can use controlled medical treatments such as medication or stenting. These interventions aim to reduce the narrowing of the artery and improve blood flow. Additionally, lifestyle modifications like exercise and a healthy diet can help improve blood flow and reduce the risk of further narrowing.

### Significant Contribution

The inventors have developed an optimization method to minimize the percentage stenosis in the arteries of the heart, PSAH, among patients with coronary artery stenosis using the desirability function method to response surface optimization methodology. The proposed optimization method presented step-by-step is very efficient and flexible to apply to optimize (minimize) the percentage stenosis of the arteries of the heart, PSAH.

The inventors have obtained an optimal (minimal) percentage stenosis of 21%, with a 95% confidence region of (11.5, 31.8) and a 95% prediction interval of (8.90, 33.1). Our analytical model has an R₂ of 96% and a high prediction accuracy of 95%. The inventors also obtained a desirability function value of 0.99≈1 which indicates that the optimal value is robust and highly efficient. These results are given in Tables 8, 9, 10, 11, 12, and 13.

The proposed optimization method is designed so that the inventors can obtain an arbitrary optimum value for the PSAH based on the desired risk factors (target) value. This allows individuals and health professionals to set their optimal value or target, and the model will search for and produce the values of the significantly identified risk factors needed to obtain the value of the PSAH at a given desirable function and 95% region of confidence, as demonstrated in Table [8].

The inventors have also developed a process of 2-D graphical visualization that displays the behavior of the percentage stenosis in the arteries of the heart, PSAH, as a function of the risk factor that influences the objective. The proposed optimization method requires a very efficient analytical model that the inventors have developed in phase I of our innovation to obtain the risk factors that will minimize the percentage stenosis in the arteries of the heart, PSAH.

Finally, knowing the interactive behavior of the contributing risk factors through the 2-D surface plots is extremely important to understanding the dynamics of the percentage stenosis. If utilized correctly, the proposed optimized method will help decrease the elevated risk, preventing cardiovascular events, such as heart attacks or strokes, before they occur. Additionally, it provides guidance for treatment decisions concerning individuals at high risk of heart disease.

### Example: Phase III—Parametric Analysis of the Percentage of Stenosis of the Artery of the Heart (PSAH)

### Overview

This innovation presents the likelihood or expectation of experiencing a cardiovascular event (such as a heart attack or stroke) at or below a specified percentage of arterial stenosis in patients with coronary artery stenosis. The medical professional considers that a percentage stenosis of the arteries of the heart of 70% or higher is of major concern. These findings are important for understanding the severity of the condition of arterial stenosis patients, so as to identify the strategy to address the subject disease. The inventors utilized real data to identify the best probability density function that characterizes the probabilistic behavior of the stenosis of the artery to be a 3-P Fatigue Life Distribution. The inventors developed the cumulative distribution function (CDF) and the survival function, along with pertinent properties associated with the subject condition. The grading scale of stenosis severity was compared with the probability findings, such as the expectation of a patient with a specific degree of stenosis. For example, the inventors want to know the probability that a given arterial stenosis patient will exceed the critical point of 70%.

### Introduction

Prior to conducting any statistical analysis and modeling, it is essential to perform a parametric analysis. Parametric analysis is the process of determining the probability distribution of the percentage stenosis under consideration. It encompasses identifying the best-fitting probability distribution that can effectively depict the data being assessed. Moreover, it assists in making decisions about whether the percentage stenosis necessitates transformation and in selecting the appropriate type of transformation to be implemented.

Parametric analysis holds a pivotal role in statistical decision analysis, as it empowers us to make well-informed decisions regarding accurate data modeling and analysis. It plays a crucial role in verifying that the assumptions associated with the chosen statistical methods are met. In a parametric test, it is assumed that the data adhere to a specific distribution, and if that probability distribution is not correct, it will lead to misleading and incorrect decisions. Thus, it is extremely important to identify the correct probability distribution that best fits the data.

Non-parametric tests are the preferred option when a unique probability distribution cannot be identified for the given data. However, it's important to note that their approach may be statistically less robust and less powerful compared to parametric analysis if a probability density function (pdf) can be determined. Conversely, if parametric analyses are applied prematurely, assuming a specific pdf, the results can be misleading.

It is important to emphasize that almost all statistical methods are based on the assumption that the given data follows the Gaussian (normal) distribution, but in our case, the inventors identified that the data was skewed and that the normal distribution is not applicable. If the inventors had assumed normality in a situation where the data was not normal, it would have led to misleading results.

### Data Description: Percentage Stenosis

The data for percentage stenosis was obtained from the PLOS Medicine Open data repository, a biomedical research cooperation in San Francisco, California, consisting of 1,132 stable and unstable angina patients who underwent invasive coronary angiography using coronary computed tomography angiography (CCTA) and/or functionally confirmed one-vessel coronary artery disease (percentage of stenosis by QCA (quantitative coronary analysis). It consists of 18 risk factors divided into the patient's clinical and angiographic characteristics. The clinical risk factors are further divided into modifiable and non-modifiable factors. Modifiable factors include smoking, hypertension, diabetes, Hyperlipidemia, BMI, and BSA. The non-modifiable factors include age, sex, family history, etc. The variable of interest under study is the percentage stenosis of the artery of the heart, PSAH.

### Percentage Stenosis of the Artery of the Heart, PSAH

As atherosclerotic plaque builds up in the arteries of a person, the inside of the arteries begins to narrow, which lessens or blocks the flow of blood. Plaque can also rupture (break open), and when this happens, a blood clot can form on the plaque, blocking the flow of blood leading to a mismatch between myocardial oxygen supply and myocardial oxygen demand and commonly resulting in ischemia. Chest pain is the most likely symptom that occurs during physical and/or emotional stress accompanied by dyspnea (shortness of breath) and fatigue.

The percentage of stenosis is a measure of the degree of narrowing of a blood vessel, usually an artery, expressed as a percentage of the vessel's original diameter. This measure is commonly used in evaluating the severity of the coronary artery disease and other arterial stenosis. For example, if a vessel's diameter is narrowed to 50% of its original size, then the percent diameter of stenosis is 50%. The percent diameter of stenosis can be determined by various imaging modalities, such as coronary angiography, computed tomography (CT), magnetic resonance imaging (MRI), ECG, and ultrasound.

Coronary Angiography is the definitive investigation and provides visualization of the affected coronaries especially prior to primary percutaneous coronary intervention. This measure is often used in clinical decision-making to determine the need for intervention or to monitor the progression of disease over time. In clinical practice, over 70% of treatment decisions still rely on visual estimation of angiographic stenosis, which has limited accuracy (about 60% to 65%) for the prediction of fractional flow reserve (FFR), a standard tool for identifying ischemia-producing coronary artery stenosis.

One of the reasons for the visual-functional mismatch is that myocardial ischemia can be affected by the supplied myocardial size, (a quantitative percent diameter) which is not always evident by coronary angiography. Although invasive coronary angiography and intravascular ultrasound (IVUS) are commonly utilized for evaluating coronary anatomy and optimizing PCI, the subjective nature of visual estimation limits the accurate estimation of stenosis severity. The majority of treatment decisions still rely on visual assessment of the degree of angiographic stenosis because of the time and expense associated with FFR-guided decision-making.

The process of interpreting complex coronary vasculature, image noise, low contrast vessels, and non-uniform illumination is time-consuming, thereby posing certain challenges to the operator. The necessity of concurrently performing noninvasive CCTA and invasive angiography has limited the clinical utility of the analytical models. The data-driven approach may support clinicians in identifying the extent and severity of complex coronary artery disease and mitigating the visual-functional mismatch between angiographic and FFR.

### Usefulness of Parametric Analysis to the Percentage of Arterial Stenosis of the Heart

1) Estimating the Percentage of Arterial Stenosis: Individuals or healthcare professionals can estimate various properties, including descriptive statistics and percentiles, of the Percentage of arterial stenosis, but the most powerful approach is through parametric analysis. This includes the expected value, a measure of central tendency representing the mean percentage of arterial stenosis, as the well as the standard deviation (SD), which indicates the variability around the mean percentage of arterial stenosis.

2) Confidence Intervals: Through parametric analysis, individuals or healthcare professionals can obtain confidence intervals with at least 95% accuracy. This implies that the actual estimate of the Percentage of Arterial Stenosis is likely to lie within the lower and upper bounds of the interval. These boundaries can be instrumental in assessing the extent of arterial stenosis required to progress to or regress from a specific stage. This information proves valuable for strategic planning and decision-making.

3) Through parametric analysis, the inventors can derive the probability density function of the identified probability distribution that characterizes the behavior of the phenomenon of interest (the Percentage of Arterial Stenosis) and visually represent it by plotting. This leads to the estimation of both the cumulative distribution function (CDF) and the reliability or survival function. The CDF enables individuals or healthcare professionals to accurately compute the probability or likelihood of a heart failure or experiencing a cardiovascular event (such as a heart attack or stroke) occurring at or before a specific percentage of arterial stenosis in a patient with coronary artery stenosis. The reliability or survival function allows for the accurate calculation of the probability or likelihood of the heart's survival, or not experiencing a cardiovascular event (heart attack or stroke), at a given percentage of arterial stenosis in a patient with coronary artery stenosis. This information aids in making well-informed decisions.

To identify the probability distribution of the phenomenon of interest, the percentage of arterial stenosis in a patient with coronary artery stenosis, the parametric analysis typically commences with the generating of a graphical representation: histogram and the presentation of descriptive statistics. The descriptive statistics offer crucial insights into the data, including measures of central tendency, variability, skewness, and kurtosis, providing a comprehensive understanding of the shape and characteristics of the percentage of arterial stenosis probability distribution. Skewness assesses the symmetry of the distribution; a negative value (less than zero) denotes left or negative skewness, while a positive value signifies right or positive skewness, [32]. Kurtosis measures the level of peakedness or flatness in the distribution; a positive value indicates a leptokurtic distribution, while a negative value implies a platykurtic distribution. A kurtosis value of zero signifies a mesokurtic distribution.

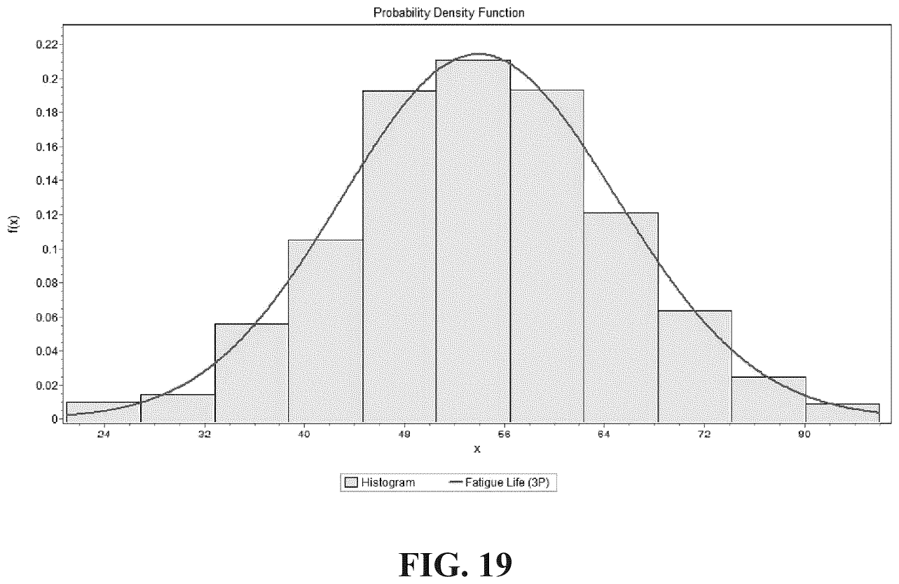

The inventors generated the histogram to investigate the distribution of the phenomenon of interest. The histogram of the Percentage of Arterial Stenosis is shown in FIG. 19.

The descriptive statistics of the Percentage of Arterial Stenosis are displayed in Table [14].

TABLE 14
Descriptive Statistics of the Percentage of Arterial Stenosis

| Sample size | Min | Max | Mean | Median | Std Err | Std Dev | Kurtosis | Skewness |
|---|---|---|---|---|---|---|---|---|
| 1132 | 21 | 86 | 54.08 | 54.00 | 0.33 | 11.01 | −0.02 | 0.03 |

From Table [14], the descriptive statistics show that the mean percentage of arterial stenosis is 54.08, which is greater than the median percentage of 54.00. This indicates that the distribution of the Percentage of arterial stenosis is right-skewed, as supported by the positive skewness value (i.e., skewness value of 0.03). The kurtosis value of −0.02 indicates a platykurtic distribution. The histogram also shows that most of the percentage values fall between 40 and 68 percent (%).

Upon careful analysis of FIG. 19 and Table [14], it was determined that the probability distribution that accurately characterizes the behavior of the percentage of arterial stenosis in patients with coronary artery stenosis follows a Three-Parameter Fatigue Life Probability Distribution, (3PFLD). Since the inventors are dealing with real-world data, it's also important to validate the distribution's fit to the phenomenon of interest (data) through statistical methods. Thus, to evaluate the validity of this probability distribution, (3PFLD), three different goodness-of-fit tests the were performed, namely the Kolmogorov-Smirnov, Anderson-Darling, and Chi-Squared tests.

### Goodness-of-Fit for the Percentage Stenosis Data

The Kolmogorov-Smirnov goodness-of-fit test is a statistical test used to determine if a set of observations is drawn from a hypothesized continuous probability distribution. It is based on the empirical cumulative distribution function (ECDF), which is a step function that jumps up by n at each observed data point. Given a dataset T={t₁, t₂, . . . , tₙ} from a continuous distribution with a cumulative distribution function (CDF), the ECDF is defined as:

$${F}_{n} ( t ) = \frac{1}{n} ( number of observations \leq t ) .$$

The Kolmogorov-Smirnov statistic is based on the largest vertical difference between the CDF and the ECDF, and is defined as:

$${D}_{n} = max \{ \vert {F}_{n} ( t ) - F ( t ) \vert \} .$$

Under the null hypothesis that the data follow the specified distribution, the distribution of the Kolmogorov-Smirnov statistic is known. The hypothesis is rejected at the a level of significance if the calculated statistic is greater than the critical value of the theoretical distribution.

Anderson-Darling test is a useful tool for assessing the goodness-of-fit of a given dataset to a specified probability distribution, especially when the tails of the distribution are of particular interest. The test is based on the comparison of the observed cumulative distribution function (CDF) to the expected CDF of the specified distribution. This test places more emphasis on the tails of the distribution than the Kolmogorov-Smirnov test. The Anderson-Darling statistic, denoted by

$${A}^{2} = - n - {\sum }_{i = 1}^{n} \frac{2 i - 1}{n} [ ln F ( {t}_{i} ) + ln ( 1 - F ( {t}_{n - i + 1} ) ) ] ,$$

is calculated as the weighted sum of the squared differences between the observed cumulative distribution function and the expected cumulative distribution function. The weights are chosen based on the inverse of the variance of the expected distribution. The statistic A² is then compared to the critical value of the theoretical distribution under the null hypothesis that the data follow the specified distribution.

The Chi-Squared goodness-of-fit test is a statistical test that requires observed data to be grouped into intervals of equal width with each group representing a cell. The expected frequencies are calculated using the cumulative distribution function of the specified distribution. The Chi-Squared statistic is calculated by comparing the observed and expected frequencies in each cell and summing the squared differences across all cells. The resulting statistic follows a Chi-Squared distribution with k−1 degrees of freedom, where k is the number of cells. There is no optimal choice for the number of cells. However, each cell is required to contain at least five data points. Given a dataset T={t₁, t₂, . . . , tₙ} with cumulative distribution function F(t), the Chi-Squared statistic denoted by χ² is defined as,

$${χ}^{2} = {\sum }_{i = 1}^{n} \frac{{( {O}_{i} - {E}_{i} )}^{2}}{{E}_{i}} ,$$

where Oᵢ is the observed frequency for cell i, and Eᵢ is the expected frequency for cell i calculated as

$${E}_{i} = F ( {t}_{2} ) - F ( {t}_{1} ) ,$$

where t₁ and t₂ are the limits for cell i. The Chi-Squared statistic is calculated under the null hypothesis that the data follow the specified distribution. The hypothesis is rejected at the level of significance if the statistic χ² is greater than the critical value X_{0.05,k-1}².

The test results revealed p-values exceeding 0.05, indicating that the null hypothesis—that the distribution of the Percentage of Arterial Stenosis follows a Three-Parameter Fatigue Life Probability Distribution—cannot be rejected. This decision is supported by all three methods.

These findings (Table 15) imply that the chosen probability distribution provides a good fit for the data and can be employed for further statistical modeling and analysis.

TABLE 15
Goodness-of-fit Test of the 3-P Fatigue Life Distribution
of the Percentage of Arterial Stenosis

| Type of Test | P-value |
|---|---|
| Kolmogorov-Smirnov | 0.4209 |
| Anderson-Darling | 0.5499 |
| Chi-Squared | 0.3052 |

Three-Parameter (3-P) Fatigue Life Probability Estimation of the Percentage of Arterial Stenosis in Patients with Coronary Artery Stenosis

Upon careful examination of FIG. 19 and descriptive statistics in Table [14], the inventors identified the best probability density function that characterizes the probabilistic behavior of the stenosis of the artery to be a 3-P Fatigue Life Distribution. Since the inventors utilized real data, it was important to validate the distribution's fit to the data through appropriate statistical methods. To validate the goodness-of-fit of this distribution, the inventors performed three distinct tests—the Kolmogorov-Smirnov, Anderson-Darling, and Chi-square tests, as detailed in Table 2. The outcomes of these tests reveal a large p-value, signifying that the inventors failed to reject the null hypothesis (H₀), suggesting that the probability distribution follows the 3-P Fatigue Life Probability Distribution.

The three-parameter fatigue life distribution is a probability distribution that describes the probability of an organ failing due to fatigue. In essence, it is used to predict the lifespan of an organ under repeated cyclic loading conditions, like the cyclic operations of the heart involving heartbeat or blood circulation. Essentially, it's a way of predicting the probability of how long something will last before it fails due to repeated stress or strain.

In this section, the inventors have defined the probability density function (pdf) for the 3-P Fatigue Life distribution and proceeded to estimate its parameters [34]. Considering the percentage of arterial stenosis in a patient with coronary artery stenosis, denoted by the random variable X, the pdf of the 3-P Fatigue Life probability distribution is given as follows

$$f ( x ; α , β , γ ) = \{ \begin{matrix}0 , & if x \leq γ \\ \frac{1}{2 β \sqrt{2 π {x}^{3}}} exp {( - \frac{{( x - γ )}^{2}}{2 {β}^{2} x} ) [ 1 + \frac{α ( x - γ )}{β} ]}^{- 3 / 2} exp ( \frac{{α}^{2} {β}^{2}}{2 ( x - γ )} ) , & if x > γ ,\end{matrix} \qquad ( 16 )$$

with the parameters having the following meanings: x∈X is the random variable, α>0 is a continuous shape parameter that affects the skewness of the distribution, β>0 is the continuous scale parameter, which controls the spread or dispersion of the distribution, y is the continuous location parameter (γ≡0 gives the two-parameter Fatigue Life distribution), which shifts the distribution along the x-axis. These parameters influence and control the probability findings of the distribution in question.

For simplicity, the general form of the probability function is expressed in terms of the standard distribution, and all subsequent formulas are provided in the standard form of the function. Thus, the probability density function (pdf) of the distribution of PSAH is given by:

$$f ( x ; α , β , γ ) = \{ \begin{matrix}0 , & if x \leq γ \\ \frac{\sqrt{\frac{( x - γ )}{β}} + \sqrt{\frac{β}{( x - γ )}}}{2 α ( x - γ )} \cdot ϕ ( \frac{1}{α} ( \sqrt{\frac{( x - γ )}{β}} - \sqrt{\frac{β}{( x - γ )}} ) ) , & if x > γ ,\end{matrix} \qquad ( 17 )$$

where φ is the probability density function of the standard normal distribution.

To estimate the three parameters, the Maximum Likelihood Estimation (NILE) procedure is employed. MLE estimates the parameters of the probability distribution by maximizing the likelihood function. Widely utilized in statistical inference, MLE is favored for its robustness compared to traditional methods such as the method of moments. It possesses unique and important statistical properties, including consistency, invariance, efficiency, sufficiency, and asymptotic normality.

To compute the NILE estimators, the first step involves determining the likelihood function.

The likelihood function for a random sample of n independent observations of the percentage of arterial stenosis in patients with coronary artery stenosis x=(x₁, x₂ . . . , xₙ) for the 3-P Fatigue Life distribution pdf,f(t) can be expressed as follows:

$$\begin{matrix}L ( γ , β , α \vert {x}_{i} ) = {\prod }_{i = 1}^{n} f ( {x}_{i} \vert γ , β , α ) \\ = {\prod }_{i = 1}^{n} [ \frac{1}{2 β \sqrt{2 π {x}^{3}}} exp {( - \frac{{( x - γ )}^{2}}{2 {β}^{2} x} ) [ 1 + \frac{α ( x - γ )}{β} ]}^{- 3 / 2} exp ( \frac{{α}^{2} {β}^{2}}{2 ( x - γ )} ) ] .\end{matrix} \qquad ( 18 )$$

Now, the inventors take the log of the likelihood function in equation (3), given by

$$\begin{matrix}ln L = ln L ( γ , β , α \vert {x}_{i} ) \\ ℓ ( γ , β , α ) = {\prod }_{i = 1}^{n} log f ( {x}_{i} ; γ , β , α ) .\end{matrix} \qquad ( 19 )$$

Substituting the PDF of the probability distribution, the inventors have:

$$ℓ ( γ , β , α ) = {\sum }_{i = 1}^{n} [ log ( \frac{1}{2 β \sqrt{2 π {x}_{i}^{3}}} ) - \frac{{( {x}_{i} - α )}^{2}}{2 {β}^{2} {x}_{i}} - \frac{3}{2} log ( 1 + \frac{α ( {x}_{i} - α )}{β} ) + \frac{{α}^{2} {β}^{2}}{2 ( {x}_{i} - α )} ] . \qquad ( 20 )$$

Next, the inventors find the partial derivatives of the log-likelihood function equation [20] with respect to each parameter.
a). Partial derivative with respect to γ:

$$\frac{\partial ℓ}{\partial γ} = {\sum }_{i = 1}^{n} [ \frac{{x}_{i} - γ}{2 {β}^{2} {x}_{i}} + \frac{3 α}{2 {β}^{2}} \frac{1}{1 + \frac{α ( {x}_{i} - γ )}{β}} - \frac{{α}^{2} {β}^{2}}{2 {( {x}_{i} - γ )}^{2}} ]$$

b). Partial derivative with respect to β:

$$\frac{\partial ℓ}{\partial γ} = {\sum }_{i = 1}^{n} [ - \frac{1}{2 β} + \frac{{( {x}_{i} - γ )}^{2}}{2 {β}^{3} {x}_{i}} + \frac{3 α}{2 β} \frac{( {x}_{i} - γ )}{{( 1 + \frac{α ( {x}_{i} - γ )}{β} )}^{2}} + \frac{{α}^{2} {β}^{2}}{2 ( {x}_{i} - γ )} ]$$

c). Partial derivative with respect to α:

$$\frac{\partial ℓ}{\partial α} = {\sum }_{i = 1}^{n} [ - \frac{3 ( {x}_{i} - γ )}{2 β ( 1 + \frac{α ( {x}_{i} - γ )}{β} )} + \frac{{α}^{2} {β}^{2}}{( {x}_{i} - γ )} ]$$

Next, the inventors set the partial derivatives equal to zero and solve for the parameters. This will give us the maximum likelihood estimates.

$$\frac{\partial ℓ}{\partial μ} = 0 , \qquad \frac{\partial ℓ}{\partial β} = 0 , \qquad \frac{\partial ℓ}{\partial α} = 0 ,$$

This step involves solving a system of equations. The inventors verify with the second derivatives by checking the second derivatives to ensure that the estimates are indeed maxima.

$$\frac{{\partial }^{2} ℓ}{\partial {μ}^{2}} , \frac{{\partial }^{2} ℓ}{\partial {β}^{2}} , \frac{{\partial }^{2} ℓ}{\partial {α}^{2}}$$

These should be negative for maxima. Based on the procedure for the parameter estimation of the 3-P Fatigue Life probability distribution discussed above, the MLEs of the parameters (γ, β, α) of the 3-P Fatigue Life distribution of the percentage of arterial stenosis in patients with coronary artery stenosis are given in Table [16] below.

TABLE 16
Parameter Estimates for the Three-Parameter Fatigue Life Probability
Distribution of the Percentage of Arterial Stenosis

| Location (\hat{γ}) | Scale (\hat{β}) | Shape (\hat{α}) |
|---|---|---|
| −908.79 | 962.83 | 0.01143 |

The inventors substitute the parameter estimates in Table [16] into Equation [16] and Equation [17] respectively to obtain the estimated probability density function (pdf) of the 3p-Fatigue Life Probability Distribution of the percentage stenosis in the artery of patients with coronary artery stenosis, given by

$$f ( x ) = \{ \begin{matrix}0 , & if Θ \text{?} \\ \begin{matrix}\frac{1}{2 ( 962.83 ) \sqrt{2 π {x}^{3}}} exp ( - \frac{{( x + 908.79 )}^{2}}{2 ( {962.83}^{2} ) x} ) \\ {[ 1 + \frac{0.01143 ( x + 908.79 )}{962.83} ]}^{- 3 / 2} exp ( \frac{{0.01143}^{2} * {962.83}^{2}}{2 ( x + 908.79 )} ) ,\end{matrix} & if Δ \text{?}\end{matrix} \qquad ( 21 )$$

$$\text{?} \text{indicates text missing or illegible when filed}$$

where Θ is if x<−908.79 and Δ is if x>−908.79.

Or The pdf expressed in the standard probability distribution form;

$$f ( x ) = \{ \begin{matrix}0 & if x \leq - 908.79 \\ \begin{matrix}\frac{\sqrt{\frac{( x + 908.79 )}{962.83}} + \sqrt{\frac{962.83}{( x + 908.79 )}}}{2 * 0.01143 ( x + 908.79 )} \cdot \\ ϕ ( \frac{1}{0.01143} ( \sqrt{\frac{( x + 908.79 )}{962.83}} - \sqrt{\frac{962.83}{( x + 908.79 )}} ) )\end{matrix} & if x > - 908.79 ,\end{matrix} \qquad ( 22 )$$

where φ is the probability density function of the standard normal distribution.

The above-estimated probability density function (pdf) findings can ensure efficient and accurate analysis of the degree of percentage arterial stenosis in the heart of patients with coronary artery stenosis.

After finding the probability density function (pdf) for the 3p-Fatigue Life Probability Distribution, the cumulative frequency distribution (CDF) can be calculated through the integration of the pdf in Equation [16] and Equation [17] with respect to the random variable X.

Using Equation [16]:

$${F}_{X} ( x ) = P ( x \leq X ) \qquad ( 23 )$$

$$\begin{matrix}{F}_{X} ( x ; γ , β , α ) = {\int }_{0}^{x} f ( x ; γ , β , α ) ds \\ = \begin{matrix}{\int }_{0}^{r} \frac{1}{2 β \sqrt{2 π {x}^{3}}} exp ( - \frac{{( x - γ )}^{2}}{2 {β}^{2} x} ) \\ {[ 1 + \frac{α ( x - γ )}{β} ]}^{- 3 / 2} exp ( \frac{{α}^{2} {β}^{2}}{2 ( x - γ )} ) ds ,\end{matrix}\end{matrix}$$

the final resulting CDF equation, Equation [24] after the complex integration process is given by:

$${F}_{X} ( x ; γ , β , α ) = 1 - {exp ( - \frac{( x - γ )}{β} ) [ 1 - \frac{α ( x - γ )}{β} ]}^{- 1 / 2} . \qquad ( 24 )$$

Substituting the parameter estimates given in Table [14], into Equation [24], then the cumulative distribution function (CDF) of a 3-Parameter Fatigue Life Probability Distribution is given by:

$${F}_{X} ( x ) = 1 - {exp ( - \frac{( x + 908.79 )}{962.83} ) [ 1 + \frac{0.01143 ( x + 908.79 )}{962.83} ]}^{- 1 / 2} . \qquad ( 25 )$$

Using Equation [17], (Expressed in the Final Useful Form is given by):

$${F}_{X} ( x ) = P ( x \leq X ) \qquad ( 26 )$$

$$\begin{matrix}{F}_{X} ( x ; γ , β , α ) = {\int }_{0}^{x} f ( x ; γ , β , α ) ds \\ = \begin{matrix}{\int }_{0}^{r} \frac{\sqrt{\frac{( x - γ )}{β}} + \sqrt{\frac{β}{( x - γ )}}}{2 α ( x - γ )} \\ ϕ ( \frac{1}{α} ( \sqrt{\frac{( x - γ )}{β}} - \sqrt{\frac{β}{( x - γ )}} ) ) ds ,\end{matrix}\end{matrix}$$

the resulting CDF equation, Equation [27] from the complex integration process is given by:

$${F}_{X} ( x ; γ , β , α ) = Φ ( \frac{1}{α} ( \sqrt{\frac{( x - γ )}{β}} - \sqrt{\frac{β}{( x - γ )}} ) ) , if x > γ \qquad ( 27 )$$

where Φ is the cumulative distribution function of the standard normal distribution.
Substituting the parameter estimates given in Table [14], into Equation [27], then the estimated cumulative distribution function (CDF) of a 3-parameter fatigue life distribution is given by:

$${F}_{X} ( x ) = Φ ( \frac{1}{0.01143} ( \sqrt{\frac{( x + 908.79 )}{962.83}} - \sqrt{\frac{962.83}{( x + 908.79 )}} ) ) . \qquad ( 28 )$$

This CDF is useful in determining that, the probability of a given heart patient percentage stenosis, x) would be less than or equal to some value x. In other words, the inventors can estimate the probability or likelihood of a patient with coronary artery stenosis experiencing a cardiovascular event (such as a heart attack or stroke) at or below a certain specified percentage of arterial stenosis of the heart.

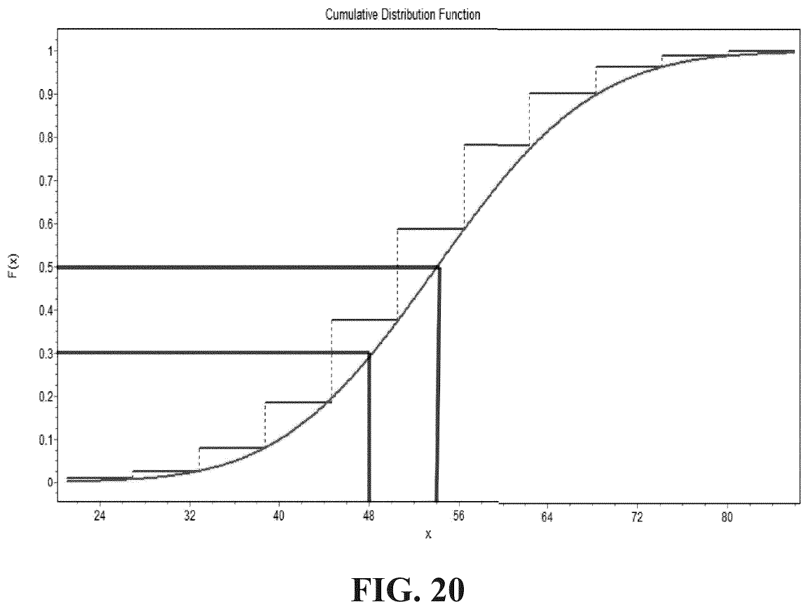

In FIG. 20, the Cumulative Distribution Function (CDF) plot for the percentage of artery stenosis is presented. By examining this plot, the inventors can estimate that the probability or likelihood of a cardiovascular event (such as a heart attack or stroke) for a patient with a coronary artery stenosis percentage of 48% and 54% is approximately 0.3 and 0.5, respectively.

From the plot, the inventors can see that, the higher the CDF, the higher the percentage of arterial stenosis, and the greater the risk of a cardiovascular event. This measure of the risk analysis is often used to guide treatment decisions for people at high risk of heart disease and to help prevent cardiovascular events before they happen.

From Table [17], the inventors can examine the grade for stenosis severity along with the probability of experiencing a cardiovascular event (heart attack or stroke) for a specified grade or percentage of stenosis.

TABLE 17
Grading Scale for Stenosis Severity
along with Probabilities and CB

| | Degree of | Percentage | | Confidence |
|---|---|---|---|---|
| Grading | Stenosis | of Stenosis | Probabilities | Bounds CB |
| No visible | 0% | 0% | 0 | — |
| stenosis | | | | |
| Minimal | 1-24% | 22% | 0.005 | (0.000, 0.005) |
| stenosis | | | | |
| Mild stenosis | 25-49% | 40% | 0.1 | (0.005, 0.350) |
| Moderate | 50-69% | 54% | 0.5 | (0.350, 0.925) |
| stenosis | | | | |
| Severe | 70-99% | 80% | 0.975 | (0.925, 0.999) |
| stenosis | | | | |
| Occluded | 100% | 100% | 1 | 1 |

Furthermore, given the Cumulative Distribution Function (CDF), of a 3-Parameter Fatigue Life Probability Distribution in Equation [27], the inventors obtained the survival function, S(t) of the 3-Parameter Fatigue Life Probability Distribution.

The survival function of the 3-parameter fatigue life distribution describes the probability of surviving without experiencing a cardiovascular event (heart attack or stroke) beyond a specified percentage of arterial stenosis in patients with coronary artery stenosis.

This allows the inventors to estimate the likelihood or probability of a patient with coronary artery stenosis surviving beyond a particular percentage of arterial stenosis in the heart.

Therefore, the estimate of the survival function of the 3-parameter fatigue life distribution,
Ŝₜ is given by

$$\begin{matrix}\hat{S} ( t ) = P ( x \geq X ) \\ = 1 - {F}_{X} ( x ) \\ = 1 - {F}_{X} ( x ; γ , β , α ) \\ = 1 - Φ ( \frac{1}{α} ( \sqrt{\frac{( x - γ )}{β}} - \sqrt{\frac{β}{( x - γ )}} ) ) ,\end{matrix} \qquad ( 29 )$$

Substituting the parameter estimates given in Table [14], into Equation [29], then the estimated survival function, \hat{ }S(t) of a 3-parameter fatigue life distribution is given by:

$$\hat{S} ( t ) = 1 - Φ ( \frac{1}{0.01143} ( \sqrt{\frac{( x + 908.79 )}{962.83}} - \sqrt{\frac{962.83}{( x + 908.79}} ) ) . \qquad ( 30 )$$

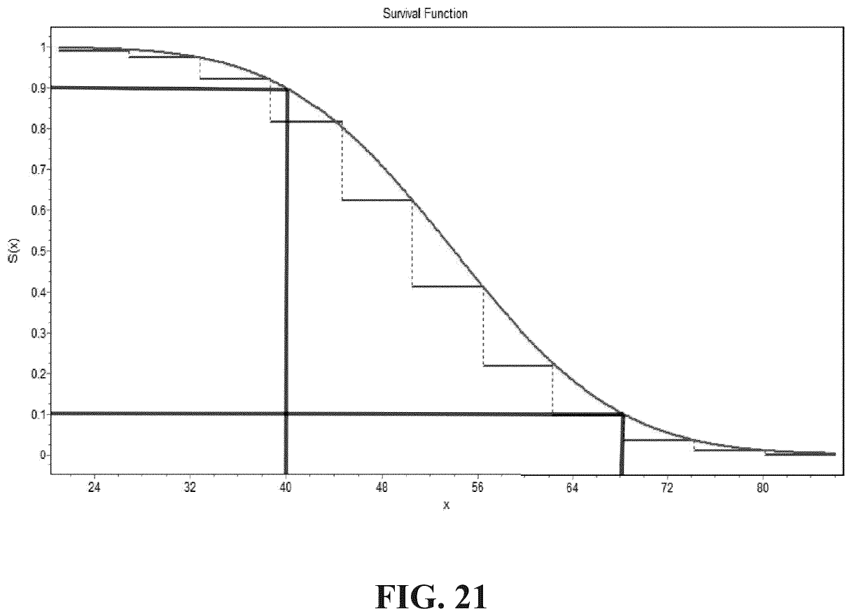

In FIG. 21, the inventors display the estimate of the survival function \hat{ }S(t) of the percentage of arterial stenosis in patients with coronary artery stenosis. 70% is the critical value of PSAH. As expected, the inventors can see that the survival estimates of percentage stenosis are decreasing and approaching approximately zero beyond the stenosis of 84%. Thus, no individual is likely to survive beyond a percentage stenosis of 84%. In other estimates, the probability that a patient survives beyond 40% and 68% is approximately 0.9 (90%) and 0.1 (10%) respectively.

### Significant Contribution

In this phase of our innovation, the inventors found the likelihood or expectation of experiencing a cardiovascular event (such as a heart attack or stroke) at or below a specified percentage of arterial stenosis in patients with coronary artery stenosis. The inventors utilized actual data to identify the best probability density function that characterizes the probabilistic behavior of the percentage stenosis of the artery to be a 3-P Fatigue Life Probability Distribution. The inventors developed its CDF and survival function. In addition, the inventors compared the grading scale of stenosis severity with our probability findings. From the results, the higher the CDF, the higher the percentage of arterial stenosis, and the greater the risk of a cardiovascular event. This measure of the risk analysis is often used to guide treatment decisions for people at high risk of heart disease and to help prevent cardiovascular events before they happen.

Below are findings from Phase III:
1) The inventors obtain the Cumulative Distribution Function, CDF. Which the inventors can use to estimate the probability or expectation of experiencing a cardiovascular event, such as a heart attack or stroke. From the plot of CDF of PSAH, the inventors can obtain useful information. For example, if the PSAH of a patient is 48%, then there is a 30% likelihood or chance that the patient would have or experience a stroke or heart attack.
2) Using the CDF of the PSAH, the inventors obtained the survival function S(t) of the 3-parameter Fatigue-Life Distribution, which gives the probability that a given patient will survive beyond a particular PSAH. From the plot of the survival function S(t), a patient with PSAH of 84% the probability of survival is close to zero, 0, while a patient with a PSAH of 40% has a 90% chance of surviving.
3) The inventors can obtain the probability that a patient will exceed the critical percentage of 70% of PSAH. Also, the inventors identified the probabilities that a given patient is in a particular stage of PSAH, extreme (severe), moderate, or mild.
4) The inventors can obtain confidence limits of true PSAH. That is, the inventors are at least 95% certain that the true value of PSAH lies between two, 2, numbers, lower and upper bound. For example, if a patient's PSAH is approximately 70% (severe stenosis), there is a 97% probability that the patient would have or experience a heart attack or stroke with confidence bounds of (0.925, 0.999).

What is claimed is:

1. A method for determining a percentage of stenosis in arteries of a heart, the method comprising:
receiving a set of patient indicators, the set comprising:
a patient's biographic indications,
a first set of patient physical exam indications,
a first indication of patient hypertension, and
a plurality of cardiovascular dimensions from patient scan data;
providing the patient indicators to a percentage stenosis analytical model;
via the stenosis analytical model, generating a first result comprising a likely percentage of arterial stenosis for the patient and a confidence interval;
providing an alert to a user based on an upper bound of the confidence interval, wherein the alert to a medical provider is transmitted if the confidence interval encompasses a percentage of arterial stenosis equal to or greater than 70%, and
providing a behavioral recommendation to the patient to lower the likely percentage of arterial stenosis if the likely percentage of arterial stenosis is greater than 50%.

2. The method of claim 1, further comprising
receiving a second set of patient physical exam indications,
wherein if the first set and second set of patient physical exam indications are equal, only one set of indications will be used in a calculation of percentage stenosis,
wherein if the first and second set of patient physical exam indications are different, the second set of patient physical exam indications are used in an updated calculation of percentage stenosis; and
receiving a second indication of patient hypertension,
wherein if the first set and second indications of patient hypertension are equal, only one set of indications will be used in the updated calculation of percentage stenosis,
wherein if the first set and second indications of patient hypertension are different, the second indication of patient hypertension is used in the calculation of percentage stenosis, and
generating a second result of arterial stenosis comprising a likely percentage of arterial stenosis for the patient and a confidence interval,
wherein the second result is generated if the second set of patient physical exam indications is different from the first set of patient physical exam indications, or if the second indication of patient hypertension is different from the first indication of patient hypertension.

3. The method of claim 1, wherein the patient biographic indications comprises: an age of the patient, and an indication of gender of the patient.

4. The method of claim 1, wherein the patient physical exam indications comprise at least one of: an indication of an indication of diabetes, an indication of hyperlipidemia, an indication of smoking history, an indication of body mass index (BMI), and an indication of body surface area (BSA).

5. The method of claim 1, wherein the indication of patient hypertension comprises a numerical value of 0 or 1,
wherein a value of 0 indicates the patient does not have high blood pressure,
wherein a value of 1 indicates the patient does have high blood pressure.

6. The method of claim 1, wherein the plurality of cardiovascular dimensions from patient scan data comprises at least one of: lesion length, calculated percentage of right coronary artery, calculated percentage of left circumflex artery, calculated percentage of left anterior descending artery, fractional flow reserve, minimum lumen diameter, distal reference lumen diameter, proximal reference lumen diameter, maximal lumen diameter within left main coronary artery segment, and distance between a ostium to the narrowest side.

7. The method of claim 1, wherein the percentage of arterial stenosis is determined by:
$${}_{p} {S}_{T} = 3.8021 - 0.4299 {X}_{1}^{′} - 2.1658 {e}^{- 02} {X}_{3} + 0.1102 {X}_{8} - 3.9886 {X}_{14} - 4.9892 {e}^{- 02} {X}_{18}^{′} - 2.1255 {e}^{- 03} {X}_{1}^{′} * {X}_{7} + 0.1649 {X}_{1}^{′} * {X}_{16} + 0.6529 {X}_{10} * {X}_{15} - 0.6194 {X}_{16} * {X}_{10} + 0.3572 {X}_{14} * {X}_{16} .$$

8. The method of claim 2, wherein the confidence interval is determined by:
$$CI = \hat{y} \pm {t}_{α / 2 , df} \cdot SE ( \hat{y} )$$
wherein t_{a/2,df} is a critical value from a t-distribution for a 95% confidence interval with degrees of freedom df, and
SE(ŷ) is a standard error of a predicted response,
wherein the standard error of the predicted response is
$$SE ( \hat{y} ) = \sqrt{{{x}^{T} ( {X}^{T} X )}^{- 1} x \cdot {σ}^{2}} .$$

9. The method of claim 1, further comprising a prediction interval, wherein the prediction interval is determined by:
$$PI = \hat{y} \pm {t}_{α / 2 , df} \cdot \sqrt{{SE}^{2} ( \hat{y} ) + {σ}^{2}}$$
where σ² is an estimated variance of residuals.

10. The method of claim 1, further comprising monitoring to determine whether new data is available for the set of patient indicators,
wherein if new data is available, determining whether the data is suitable for use in recalculating the likely percentage of arterial stenosis by evaluating a length of time between a collection of new data and original data,
whereby if the length of time is more than four months, the new data is used in recalculating the likely percentage of arterial stenosis via the stenosis analytical model; and providing a second result to the patient.

11. The method of claim 4, further comprising monitoring to determine whether new data is available for the indication of body mass index (BMI) or the indication of body surface area,
wherein if new data is available, determining whether the data is suitable for use in recalculating the likely percentage of arterial stenosis by evaluating if the indication of body mass index or the indication of body surface area has a 5% or greater change for the patient,
whereby if the change of body mass index or the indication of body surface area has changed by 5% or more, the indication of body mass index and the indication of body surface area obtained more recently is used in recalculating the likely percentage of arterial stenosis via the stenosis analytical model; and
providing a second result to the patient.

12. The method of claim 3, further comprising recalculating the likely percentage of arterial stenosis using an updated age of the patient if the age of the patient has increased by at least one year.

13. The method of claim 2, further comprising rejecting the second result if the second set of patient physical exam indications or the second indication of hypertension have greater than a twenty percent change within six months of obtaining the first set of patient physical exam indications or the first indication of hypertension.

14. The method of claim 1, further comprising a recommendation to the patient to adopt the behavioral recommendation and recalculate the likely percentage of arterial stenosis within four months if the first result of percentage of arterial stenosis is greater than 70%.

15. The method of claim 2, further comprising if the second set of patient physical exam indications are significantly worse than the first set of patient physical exam indications, or if the second indications of patient hypertension are significantly worse than the first indication of patient hypertension, a warning is provided to the patient describing potential health outcomes and interventions needed to reduce the percentage of arterial stenosis.

16. The method of claim 15, further comprising providing the patient a communication encouraging the patient how the percentage of arterial stenosis can decrease with appropriate interventions.

17. A method for determining a percentage of stenosis in arteries of a heart, the method consisting of:
receiving a set of patient indicators, the set consisting of:
a patient's biographic indications,
a first set of patient physical exam indications,
a first indication of patient hypertension, and
a first plurality of cardiovascular dimensions from patient scan data;
providing the patient indicators to a percentage stenosis analytical model;
wherein the percentage stenosis analytical model incorporates a measurement of minimal lumen diameter, an interaction between the minimal lumen diameter and a proximal reference lumen diameter, an interaction between a calculated percentage of a right coronary artery and distal reference lumen diameter, an interaction between a calculated percentage of the right coronary artery and a proximal reference lumen diameter, an age of the patient, an indication of body surface area, an interaction between age and an indication of body mass index, a distance to ostium from minimal lumen diameter, and the first indication of patient hypertension,
via the stenosis analytical model, generating a first result comprising a likely percentage of arterial stenosis for the patient and a confidence interval;
providing an alert to a user based on an upper bound of the confidence interval, wherein the alert to a medical provider is transmitted if the confidence interval encompasses a percentage of arterial stenosis equal to or greater than 70%, and
providing a behavioral recommendation to the patient to lower the likely percentage of arterial stenosis if the likely percentage of arterial stenosis is greater than 50%.
